(19) United States
(12) Patent Application Publication
(10) Pub. No.: US 2025/0250613 A1
(43) Pub. Date: Aug. 7, 2025

(54) DISCOVERY OF BACTERIAL AIEGEN THAT LIGHTS UP SPECIFIC G-QUADRUPLEXES

(71) Applicants: Southern Marine Science and Engineering Guangdong Laboratory (Guangzhou), Guangzhou (CN); The Hong Kong University of Science and Technology, Hong Kong (CN)

(72) Inventors: Pei-Yuan QIAN, Hong Kong (CN); Wenkang YE, Hong Kong (CN); Ben Zhong TANG, Hong Kong (CN); Xinnan WANG, Hong Kong (CN); Changdong LIU, Hong Kong (CN); Ching SUEN, Hong Kong (CN); Guang ZHU, Hong Kong (CN); Ian Duncan WILLIAMS, Hong Kong (CN); Parvej ALAM, Hong Kong (CN)

(21) Appl. No.: 19/047,732

(22) Filed: Feb. 7, 2025

Related U.S. Application Data
(60) Provisional application No. 63/550,624, filed on Feb. 7, 2024.

(51) Int. Cl. C12Q 1/6816; G01N 21/64

(52) U.S. Cl. CPC C12Q 1/6816; G01N 21/6428; G01N 2021/6439

(57) ABSTRACT

The subject invention pertains to a method for identifying G-quadruplexes (G4s) in a cell using a G4 fluorescent probe, 2-(2-hydroxy-6-methoxy-3-propionylphenyl)quinazolin-4(3H)-one (HMPQ), which exhibits specific binding affinity to G-quadruplexes (G4s), illuminating these non-canonical nucleic acid structures without altering their conformation. G4s are involved in chromatin organisation, gene regulation, genome stability, and appear to contribute to cancer growth and progression. By utilizing HMPQ's unique fluorescence mechanism through excited-state intramolecular proton transfer, the method allows label-free precise detection of G4 structures in the nucleus of a cell.

### CROSS-REFERENCE TO RELATED APPLICATION

This application claims the benefit of U.S. Provisional Application Ser. No. 63/550,624, filed Feb. 7, 2024, which is hereby incorporated by reference in its entirety including any tables, figures, or drawings.

### SEQUENCE LISTING

The Sequence Listing for this application is labeled “HKUS-200X-SeqList.xml” which was created on Nov. 21, 2024 and is 19,256 bytes. The entire contents of the sequence listing is incorporated herein by reference in its entirety.

### BACKGROUND OF THE INVENTION

G-quadruplexes (G4s) are structures that have garnered attention as potential therapeutic targets owing to their implicated roles in chromatin organization, gene regulation, and genome stability, as well as their contribution to treating various human diseases^{1,2,3}. Despite the design of numerous G4 ligands aimed at modulating these biological switches⁴, a comprehensive understanding of the exact nature of G4s remains elusive. The challenge persists primarily because intracellular G4s, particularly those within the nucleus, are difficult to detect and analyze primarily owing to the absence of efficient probes.

The discovery of the aggregation-induced emission (AIE) phenomenon^{5,6,7} has led to the identification and design of numerous small molecules and polymeric materials with AIE properties, often called AIE luminogens (AIEgens). Distinguished by their exceptional optical performance, these materials have widespread applications across diverse domains, including bioimaging, optoelectronic devices, and biochemical sensors^{8,9,10,11}. Nevertheless, the chemical synthesis of AIEgens encounters challenges such as limited structural diversity, restricted physicochemical properties, environmental concerns, elevated costs, and uncertain biocompatibility¹². Conversely, natural products have emerged as compelling alternatives, characterized by enhanced biocompatibility and a wealth of bioactive properties. Natural products' intricate structures and chemical diversity have inspired chemists in recent decades¹³. Notably, some AIEgens such as curcumin¹⁴, quercetin¹⁵, berberine¹⁶, and coumarin¹⁷ have been isolated from plants.

Alternatively, small molecules produced by bacteria offer many advantages, including shorter life cycles, suitability for large-scale fermentation, biosynthetic pathways that are amenable to deciphering and engineering, and relatively straightforward purification processes. However, the exploration of bacterial metabolites, another abundant reservoir of natural resources in the realm of optical activity and luminescent materials, remains relatively limited^{18,19,20}. Yet, bacterial metabolites from the unique high-salt, high-pressure, and low-temperature marine environment are attractive sources of drug leads and biochemicals²¹. Therefore, given the biological and medical importance of G4s, there is an urgent need for developing an efficient method that utilizes a highly specific and easy to obtain AIEgen probe for exploring G4s within cells.

### BRIEF SUMMARY OF THE INVENTION

The subject invention discloses a label-free method for identifying G4s within a cell or the nucleus of a cell. In embodiments, the method comprises obtaining a fluorescent probe that selectively binds to G4s structures in cells comprising nucleic acids. In embodiments, the fluorescent probe contacts the nucleic acids in the cells and when the fluorescent probe binds to a G-quadruplex structure, the probe emits a fluorescence signal that can be detected and analyzed, thus allowing the precise identification of G4s in the cells.

In embodiments, the fluorescent probe includes, but is not limited to, 2-(2-hydroxy-6-methoxy-3-propionylphenyl)quinazolin-4(3H)-one (HMPQ). In preferred embodiments, the fluorescent probe is HMPQ. In more preferred embodiments, the HMPQ utilized includes highly purified HMPQ. In embodiments, the degree of purity of HMPQ comprises at least DNA grade, proteomics grade, molecular biology grade, or ultra-pure grade, depending on the specific technique or type of experiment for which HMPQ is utilized. Selecting the purity grade of HMPQ is well within the skill level of a person of ordinary skill in the art. In most preferred embodiments, the HMPQ utilized is a conformer type 2 polymorphic form.

In embodiments, HMPQ selectively binds to G4 structures in the cell with a peak emission wavelength of from about 497 to about 500 nm. In embodiments, HMPQ can bind to G4s without altering their conformation. Additionally, in certain embodiments, HMPQ is not cytotoxic_towards mammalian cells and cell lines.

In embodiments, HMPQ is compatible with use in aqueous media. In embodiments, HMPQ binding to G4s is detectable in subcellular compartments, including, but not limited to, the nucleus, cytoplasm, and mitochondria.

### BRIEF DESCRIPTION OF THE DRAWINGS

FIGS. 1A-1E—FIGS. 1A-1C illustrate the fermentation, isolation, and structural elucidation of the natural bacterial metabolite AIE compound, HMPQ. FIG. 1A illustrates the fermentation of the natural bacterial metabolite AIE compound. FIG. 1B illustrates the Isolation of the natural bacterial metabolite AIE compound. FIG. 1C shows a structural elucidation of the natural bacterial metabolite AIE compound. FIG. 1D illustrates fluorescence images of HMPQ's crystal samples taken under 365 nm UV light irradiation. FIG. 1E illustrates a normalized absorption spectrum of HMPQ in solid film and its photoluminescence spectrum in crystal state. Excitation wavelength: 320 nm.

FIGS. 2A-2C—FIG. 2A shows photographs of HMPQ in different solvent systems taken under 365 nm UV irradiation from a hand-held UV lamp. FIG. 2B illustrates the enol-ketone transformation during ESIPT. FIG. 2C shows normalized photoluminescence (PL) spectra of HMPQ in different solvent systems. (excitation wavelength: 340 nm; solution concentration: 10 μM).

FIGS. 3A-3D—FIG. 3A shows the photoluminescence (PL) spectrum, FIG. 3B the relative PL intensity and emission peak wavelength, and FIG. 3C the quantum yield of the film made with different weight percentages of HMPQ in PMMA mixtures. FIG. 3D illustrates the quantum yield of HMPQ in different sample states. The films were made with HMPQ and PMMA mixtures, where wt. % signifies the weight percentage of HMPQ (excitation wavelength: 320 nm).

FIGS. 4A-4D—FIGS. 4A and 4C show space-filling models of the twisted and planar polymorphs and their molecular packings. FIG. 4B shows the crystal morphology of HMPQ with different conformers exhibiting different photoluminescence quantum yields under UV light (excitation wavelength: 385 nm). FIG. 4C shows space-filling models of the planar polymorphs and their molecular packings. FIG. 4D shows the DFT analysis of HMPQ's two conformers' Gibbs free energy calculated at the M062x/TZVP level of theory in the IEFPCM model (methanol).

FIGS. 5A-5E—FIG. 5A shows photoluminescence (PL) spectra of HMPQ mixed with different G4s solutions (50 μM, 400 μL) in 20 mM KH₂PO₄ buffers (70 mM KCl, 10% D₂O, pH 7.0). FIG. 5B shows PL spectra of HMPQ in buffer containing different amounts of c-kit2. FIG. 5C shows Stern-Volmer plots of relative intensity (I/I₀) versus the c-kit2 concentration. I₀=PL intensity in the absence of c-kit2. FIG. 5D shows fluorescence images taken under 365 nm UV light irradiation. FIG. 5E shows graphical abstracts of the working mechanism of HMPQ lighting up G4s but not other oligonucleotides.

FIGS. 6A-6C—FIG. 6A shows an NMR titration of HMPQ with c-kit2 G4 at different molar ratios: 0.05 mM c-kit2 and 20 mM KH₂PO₄ buffers (70 mM KCl, 10% D₂O, pH 7.0). FIG. 6B shows a circular dichroism (CD) spectrum of HMPQ with c-kit2 G4 in the same buffers in which the (CD) spectrum affirms its non-disruptive nature towards the original conformation of c-kit2 G4. FIG. 6C shows confocal images of HeLa cells treated with HMPQ and where G4s are detected by the G4 structure-specific antibody, BG4. Overlap coefficient=0.943.

FIG. 7 shows HRMS spectrum of HMPQ (C₁₈H₁₅N₂O₄⁻, Calc: 323.1037, found 323.1035, 0.62 ppm)

FIGS. 8A and 8B illustrate the HPLC profile (30% acetonitrile-water isocratic flow, observed under 254 nm) and UV spectrum of HMPQ, respectively.

FIG. 9 illustrates the ¹H NMR spectrum of HMPQ (500 MHz, DMSO-d₆). ¹H NMR (500 MHz, DMSO-d₆) δ 12.97 (s, 1H), 12.41 (s, 1H), 8.16 (dd, J=7.9, 1.6 Hz, 1H), 8.14 (d, J=9.1 Hz, 1H), 7.83 (ddd, J=8.5, 7.2, 1.6 Hz, 1H), 7.70-7.63 (m, 1H), 7.55 (ddd, J=8.1, 7.1, 1.2 Hz, 1H), 6.81 (d, J=9.1 Hz, 1H), 3.86 (s, 3H), 3.12 (q, J=7.2 Hz, 2H), 1.12 (t, J=7.2 Hz, 3H).

FIG. 10 illustrates the ¹³C NMR spectrum of HMPQ (126 MHz, DMSO-d₆).

¹³C NMR (126 MHz, DMSO-d₆) δ 206.26, 162.82, 161.79, 160.94, 149.06, 148.84, 134.46, 134.28, 127.19, 126.84, 125.81, 121.25, 113.60, 111.80, 103.15, 56.46, 31.11, 8.29.

FIG. 11 illustrates the COSY spectrum of HMPQ (500 MHz, DMSO-d₆).

FIG. 12 illustrates the HSQC spectrum of HMPQ (500 MHz, DMSO-d₆).

FIG. 13 illustrates the HMBC spectrum of HMPQ (500 MHz, DMSO-d₆).

FIG. 14 illustrates the normalized absorption spectra of HMPQ in solution and film state. Concentration of solutions: 10 μM.

FIG. 15 illustrates the molecular packing of conformer type 1.

FIG. 16 illustrates the intermolecular and intramolecular interaction of conformer type 1.

FIG. 17 illustrates the molecular packing of conformer type 2.

FIG. 18 illustrates the intermolecular and intramolecular interaction of conformer type 2.

FIG. 19 illustrates the NMR titration of HMPQ with c-myc (SEQ ID NO: 1) at different molar ratios: 0.05 mM c-myc and 20 mM KH₂PO₄ buffer (70 mM KCl,10% D₂O, pH 7.0).

FIG. 20 illustrates the NMR titration of HMPQ with c-kit1 (SEQ ID NO: 2) at different molar ratios: 0.05 mM c-kit1 and 20 mM KH₂PO₄ buffer (70 mM KCl,10% D₂O, pH 7.0).

FIG. 21 illustrates the NMR titration of HMPQ with c-kit2 (SEQ ID NO: 3) at different molar ratios: 0.05 mM c-kit2 and 20 mM KH₂PO₄ buffer (70 mM KCl,10% D₂O, pH 7.0).

FIG. 22 illustrates the NMR titration of HMPQ with htel23 (SEQ ID NO: 4) at different molar ratios: 0.05 mM htel23 and 20 mM KH₂PO₄ buffer (70 mM KCl,10% D₂O, pH 7.0).

FIG. 23 illustrates the NMR titration of HMPQ with htel21_T18 (SEQ ID NO: 5) at different molar ratios: 0.05 mM htel21_T18 and 20 mM KH₂PO₄ buffer (70 mM KCl,10% D₂O, pH 7.0).

FIG. 24 illustrates the NMR titration of HMPQ with (G4C2)4 (SEQ ID NO: 6) at different molar ratios: 0.05 mM (G4C2)4 and 20 mM KH₂PO₄ buffer (70 mM KCl,10% D₂O, pH 7.0).

FIG. 25 illustrates the NMR titration of HMPQ with r(G4C2)2 (SEQ ID NO: 7) at different molar ratios: 0.05 mM r(G4C2)2, 20 mM KH₂PO₄ buffer (70 mM KCl,10% D₂O, pH 7.0).

FIG. 26 illustrates the NMR titration of HMPQ with rTerra (SEQ ID NO: 8) at different molar ratios: 0.05 mM rTerra and 20 mM KH₂PO₄ buffer (70 mM KCl,10% D₂O, pH 7.0).

FIG. 27 illustrates the NMR titration of HMPQ with double strand DNA G4 (SEQ ID NO: 9) at different molar ratios: 0.05 mM dsDNA and 20 mM KH₂PO₄ buffer (70 mM KCl,10% D₂O, pH 7.0).

FIG. 28 illustrates the NMR titration of HMPQ with single strand DNA G4 (SEQ ID NO: 10) at different molar ratios: 0.05 mM dsDNA and 20 mM KH₂PO₄ buffer (70 mM KCl,10% D₂O, pH 7.0).

FIG. 29 illustrates the CD spectra of c-kit1 G4 before and after adding HMPQ. The CD spectrum affirms its non-disruptive nature towards the original conformation of c-kit1 G4.

FIG. 30 illustrates the circular dichroism spectra of c-myc G4 before and after adding HMPQ. The CD spectrum affirms its non-disruptive nature towards the original conformation of c-myc G4.

BRIEF DESCRIPTION OF THE SEQUENCES

| | |
|---|---|
| SEQ ID NO: 1 | TGA GGG TGG GTA GGG TGG GTA A (c- |
| | myc) DNA G4 |
| SEQ ID NO: 2 | AGG GAG GGC GCT GGG AGG AGG G (c- |
| | kit1) DNA G4 |
| SEQ ID NO: 3 | CGG GCG GGC GCG AGG GAG GGG (c- |
| | kit2) DNA G4 |
| SEQ ID NO: 4 | TAG GGT TAG GGT TAG GGT TGG GG |
| | (hte123) DNA G4 |
| SEQ ID NO: 5 | GGG AGG CGT GGC CTG GGC GGG ACT |
| | GGG G (LTR-III) DNA |
| SEQ ID NO: 6 | GGG TTA GGG TTA GGG TTT GGG |
| | (hte121 T18) DNA G4 |
| SEQ ID NO: 7 | GGG GCC GGG GCC GGG GCC GGG GCC |
| | (G4C2)4 DNA G4 |
| SEQ ID NO: 8 | GGG GCC GGG GCC (G4C2)2 RNA G4 |
| SEQ ID NO: 9 | UAG GGU UAG GGU (Terra) RNA G4 |
| SEQ ID NO: 10 | GCT TTA AAA AGT AAG TT (AT-rich) |
| | ds DNA |
| SEQ ID NO: 11 | TTC GCG CGC GTT TTC GCG CGC G |
| | (ds22) ds DNA |
| SEQ ID NO: 12 | GCG CGC GCG CGC GCG C (d(GC)8) |
| | ds DNA |
| SEQ ID NO: 13 | CTA GGG CCT AG (ds11) ds DNA |
| SEQ ID NO: 14 | GGC CCT TTT TTT TCT AG (T-rich) |
| | ss DNA |
| SEQ ID NO: 15 | CCT TCC CCA CCC TCC CCA CCC TCC |
| | CCA (s-myc) ss DNA |
| SEQ ID NO: 16 | CCC TAA CCC TAA CCC TAA CCC T |
| | (s-Tel) ss DNA |
| SEQ ID NO: 17 | GCG CGC GCG CGC GCG C (Z-DNA) ss |
| | DNA |
| SEQ ID NO: 18 | CGG GCG GGC GCG AGT GAG GGG (c- |
| | kit2_T15) ss DNA |
| SEQ ID NO: 19 | CGC GGT GTC CGC G (DNA hairpin) |
| | ss DNA |
| SEQ ID NO: 20 | GGA GAU CGC ACU CCA (RNA hairpin) |
| | ss RNA |

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts, the term “about” is providing a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. The term nucleic acid is used interchangeably with gene, cDNA, and mRNA encoded by a gene.

As used herein, the terms “oligo”, “oligonucleotide” are used interchangeably to describe short single strands of synthetic DNA or RNA, such as, for example, about a 5 nucleic acid base sequence to about a 500 nucleic acid base sequence.

As used herein, the term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

In this application, the terms “polypeptide”, “peptide”, and “protein” are used interchangeably herein to refer to a polymer of amino acids. The terms apply to amino acid polymers in which one or more amino acid residues are artificial chemical mimetic of a corresponding naturally occurring amino acids, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “label” and like terms refer to a composition detectable by spectroscopic, photochemical, biochemical, immunochemical, chemical, or other physical means. For example, labels include fluorescent dyes (fluorophores), luminescent agents, electron-dense reagents, enzymes (e.g., as commonly used in an ELISA), biotin, enzymes acting on a substrate (e.g., horseradish peroxidase), digoxigenin, ³²P and other isotopes, haptens, and proteins which can be made detectable, e.g., by incorporating a fluorescent label into the peptide or used to detect antibodies specifically reactive with the peptide. The term includes combinations of single labeling agents, e.g., a combination of fluorophores that provides a unique detectable signature, e.g., at a particular wavelength or combination of wavelengths. In the context of detecting nucleic acids (e.g., target sequences), the probes of the subject invention are fluorescent and therefore are label-free, i.e., do not need a secondary label.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

The term “organism” as used herein includes viruses, bacteria, fungi, plants and animals. Additional examples of organisms are known to a person of ordinary skill in the art and such embodiments are within the purview of the materials and methods disclosed herein. The assays described herein can be useful in analyzing any genetic material obtained from any organism.

The term “genome”, “genomic”, “genetic material” or other grammatical variation thereof as used herein refers to genetic material from any organism. A genetic material can be viral genomic DNA or RNA, nuclear genetic material, such as genomic DNA, or genetic material present in cell organelles, such as mitochondrial DNA or chloroplast DNA. It can also represent the genetic material coming from a natural or artificial mixture or a mixture of genetic material from several organisms.

As used herein, “a target nucleic acid” or “a target sequence” is a G4 or G4 structure in the genetic material of an organism.

The term “hybridizes with” when used with respect to two sequences indicates that the two sequences are sufficiently complementary to each other to allow nucleotide base pairing between the two sequences. Sequences that hybridize with teach other can be perfectly complementary but can also have mismatches to a certain extent. Therefore, the sequences at the 5′ and 3′ ends of the extension and ligation probes described herein may have a few mismatches with the corresponding target sequences at the 5′ and 3′ ends of the target genomic region as long as the extension and the ligation probes can hybridize with the target sequences to facilitate capturing of the target genomic region. Depending upon the stringency of hybridization, a mismatch of up to about 5% to 20% between the two complementary sequences would allow for hybridization between the two sequences. Typically, high stringency conditions have higher temperature and lower salt concentration and low stringency conditions have lower temperature and higher salt concentration. High stringency conditions for hybridization are preferred, and therefore, the sequences at the 3′ and 5′ ends of the extension and ligation probes are preferred to be perfectly complementary to the corresponding target sequences at the 3′ and 5′ ends of the target genomic region.

Also, two sequences that correspond to each other, for example, a target sequence and a primer sequence, have at least 90% sequence identity, preferably, at least 95% sequence identity, even more preferably, at least 97% sequence identity, and most preferably, at least 99% sequence identity, over at least 70%, preferably, at least 80%, even more preferably, at least 90%, and most preferably, at least 95% of the sequences. Alternatively, two sequences that correspond to each other are reverse complementary to each other and have at least 90% perfect matches, preferably, at least 95% perfect matches, even more preferably, at least 97% perfect matches, and most preferably, at least 99% perfect matches in the reverse complementary sequences, over at least 70%, preferably, at least 80%, even more preferably, at least 90%, and most preferably, at least 95% of the sequences. Thus, two sequences that correspond to each other can hybridize with each other or hybridize with a common reference sequence over at least 70%, preferably, at least 80%, even more preferably, at least 90%, and most preferably, at least 95% of the sequences. Preferably, two sequences that correspond to each other are 100% identical over the entire length of the two sequences or 100% reverse complementary over the entire length of the two sequences.

Any compositions or methods provided herein can be combined with one or more of any of the other compositions and methods provided herein.

Other features and advantages of the invention will be apparent from the following description of the preferred embodiments thereof, and from the claims.

All references cited herein are hereby incorporated by reference in their entirety.

The subject invention pertains to a novel label-free method for detecting G-quadruplexes (G4s) in a cell utilizing AIEgens. In embodiments, the method utilizes an AIEgen fluorescent probe that selectively binds to G4s. In embodiments, the method utilizes an AIEgen fluorescent that detects G4s without the need of utilizing a secondary label. In preferred embodiments, the AIEgen utilized as a probe is 2-(2-hydroxy-6-methoxy-3propionylphenyl)quinazolin-4(3H)-one (HMPQ), having formula (I):

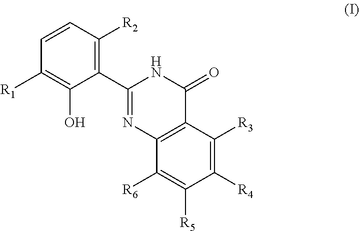

where R₁-R₆ are independent of one another and are selected from the group consisting of:
(a) alkyl;
(b) alkenyl;
(c) alkynyl;
(d) halogen-substituted alkyl;
(e) hydroxyl (—OH);
(f) ether bond (—O—);
(g) carbonyl:
(h) carboxyl (—COOH);
(i) ester (—COOR);
(j) amino (—NH₂);
(k) nitro (—NO₂);
(l) sulfonic acid (—SO₃H); and
(m) mercapto (—SH).

In certain embodiments, alkyl group comprises at least one of methyl (—CH₃), e.g., toluene (C₆H₅—CH₃), ethyl (—C₂H₅ or —CH₂CH₃), e.g., ethylbenzene (C₆H₅—CH₂CH₃), propyl (—C₃H₇), e.g., n-propyl (—CH₂CH₂CH₃) and isopropyl (—CH(CH₃)₂), or butyl (—C₄H₉), e.g., n-butyl (—CH₂CH₂CH₂CH₃), sec-butyl (—CH₂CH(CH₃)₂), isobutyl (—CH₂CH₂(CH₃)) and tert-butyl (—C(CH₃)₃).

In certain embodiments, alkenyl group comprises at least one of vinyl (—CH═CH₂), e.g., monomer vinyl chloride (CH₂═CHCl) of polyvinyl chloride (PVC), propenyl (—CH₂—CH═CH₂) or allyl (—CH═CH—CH₃), e.g., allyl alcohol (CH₂═CH—CH₂OH).

In certain embodiments, alkynyl group comprises ethynyl (—C≡CH), e.g., acetylene (C₂H₂),

In certain embodiments, halogen-substituted alkyl group comprises at least one of fluorine-substituted (—CH₂F, —CF₃), e.g., trifluoromethane (CHF₃), chlorine-substituted (—CHCl, —CCl₃, etc.), e.g., chloroform (CHCl₃), bromine-substituted (e.g., —CH₂Br, —CBr₃), or iodine-substituted (e.g., —CH₂I, —CI₃).

In certain embodiments, hydroxyl group (—OH) comprises at least one of alcohols, e.g., CH₃OH, ethanol C₂H₅OH, or phenols (e.g., C₆H₅OH).

In certain embodiments, ether bond group (—O—) comprises at least one of CH₃—O—CH₃ or diethyl ether (C₂H₅—O—C₂H₅).

In certain embodiments, carbonyl group comprises aldehyde (—CHO), e.g., formaldehyde (HCHO) and acetaldehyde (CH₃CHO).

In certain embodiments, ketone group (—CO—) comprises, for example, acetone (CH₃—CO—CH₃)

In certain embodiments, carboxyl group (—COOH) comprises, for example, formic acid (HCOOH), acetic acid CH₃COOH).

In certain embodiments, ester group (—COOR) comprises, for example, ethyl acetate (CH₃COOC₂H₅).

In certain embodiments, amino group (—NH₂) comprises amine compounds (e.g., methylamine (CH₃NH₂)).

In certain embodiments, nitro (—NO₂) comprises nitro compounds (e.g., nitrobenzene (C₆H₅NO₂)).

In certain embodiments, sulfonic acid group (—SO₃H) comprises sulfonic acid compounds.

In certain embodiments, mercapto group (—SH) comprises thiol compounds (e.g., ethanethiol (C₂H₅SH)).

In a preferred embodiment, provided herein is synthetic HMPQ homologue 310, wherein R1, and R3-R6 are hydrogen atoms and R2 is a hydroxyl group (—OH). In some embodiments, HMPQ homologue 310 emits blue light under ultraviolet light.

Conventional fluorescent probes rely on hydrophobic aromatic rings and π-conjugated chromophores²². However, their tendency to aggregate in hydrophobic pockets upon binding to nucleic acid strands in aqueous environments often results in the quenching of emitted light. Conversely, AIEgens excel in aqueous media, evading aggregation-caused quenching. In preferred embodiments, photoluminescence and NMR titration experiments unequivocally demonstrate the selective binding of HMPQ molecules to specific G4s whilst showing no interaction with regular double-stranded or single-stranded, or hairpin-structure nucleotides. In embodiments, binding to G4s restricts the mobility of HMPQ molecules, leading to a significant enhancement in fluorescence intensity. CD spectra confirm that HMPQ maintains the original conformation of G4s and its high biocompatibility enables visualization of G4s within the cellular nucleus. In embodiments, the method of the subject invention notably expands the utility of AIE-active molecules in G4 staining, an area with limited reported AIEgens²³, and allows the visualization of illuminated G4s within cells without the need for secondary labeling.

In certain embodiments, HMPQ is applicable to live cells, as previously described²⁴. In embodiments, nanoparticles containing HMPQ can be used to detect G4s in live cells utilizing confocal microscopy.

In certain embodiments, HMPQ is the first BioAIEgen isolated from bacterial metabolites through meticulous single-crystal structural analysis and rigorous photophysical testing. The fluorescence of HMPQ arises from the restriction of intramolecular and intermolecular motions⁷, and HMPQ exhibits an inherent feature known as excited-state intramolecular proton transfer (ESIPT)²⁶. ESIPT results in a substantial Stokes shift, and this characteristic renders HMPQ an exemplary candidate small-molecule fluorescent probe. In preferred embodiments, HMPQ's high biocompatibility further facilitates the visualization of G4s within the cellular nucleus.

In preferred embodiments, HMPQ displays specific binding affinity to G4s, non-canonical secondary structures found in guanine-rich regions of DNA or RNA, such as telomeres, promoters, and UTRs^{27,28}. These structures are important as potential therapeutic targets.

In one instance, the term “quencher” refers to a substance which reduces emission from a fluorescent donor when in proximity to the donor. In preferred embodiments, the quencher is within 10, 11, 12, 13, 14, 15, 16, 17, 18, 19, 20, 21, 22, 23, 24, 25, 26, 27, 28, 29 or 30 nucleotide bases of the fluorescent label. Fluorescence is quenched when the fluorescence emitted from the fluorophore is detectably reduced, such as reduced by 10%, 20%, 30%, 40%, 50%, 60%, 70%, 80%, 90%, 95%, 99% or more.

In certain embodiments, the concentration of the fluorescent probe in the compositions and method of use is about 0.01 μM to about 1000 μM, about 0.1 μM to about 100 μM, about 0.1 μM to about 50 μM, about 0.1 μM to about 10 μM, or about 0.5 μM to about 3 μM. In certain embodiments, the concentration of the fluorescent probe is about 0.01 μM, about 0.1 μM, 0.2 μM, about 0.25 μM, about 0.3 μM, about 0.4 μM, about 0.5 μM, about 0.6 μM, about 0.7 μM, about 0.8 μM, about 0.9 μM, about 1 μM, about 1.5 μM, about 2 μM, about 2.5 μM, or about 3 μM.

In certain embodiments, HMPQ displays fluorescence around 500 nm with the advantage of being in the visible spectrum and capable of being detected using existing photomultiplier tubes. In some embodiments, the visual fluorescence signal can be seen by the naked eye or taken a picture of by smartphone.

In certain embodiments, the composition of the subject invention can contain other compounds, such as antibodies, fluorophores, oligonucleotides, preservatives, buffers, and any combination thereof. These compounds can be added to the composition can be included in the composition at 0.01 to 99.9%, 0.1 to 90%, 0.5 to 80%, 0.75 to 70%, 1.0 to 50%, 1.5 to 25%, or 2.0 to 15% by weight, with respect to the total composition.

### MATERIALS AND METHODS

### Method

### General Information

Streptomyces chrestomyceticus BCC24770 was acquired from the Thailand Bioresource Research Centre. All oligonucleotides used were bought from Integrated DNA Technologies company (Singapore). NMR spectra were recorded on a 500 M or 800 M Varian spectrometer at 25° C. HRMS data were recorded on a Xevo G2-XS QTOF mass spectrometer from Water's. HPLC analysis or preparation was performed using a Waters HPLC system (Waters 2695 Separations Module; Milford, USA) and monitored under a Waters 2998 Photodiode Array Detector. Semi-preparative HPLC was performed using a Phenomenex Luna C18 column (250 mm×10 mm in size) and designed for a particle size of 5 μm. Single-crystal XRD data were collected on a Rigaku-Oxford Diffraction Supernova Dual Atlas diffractometer. Crystal photos were taken under Nikon ECLIPSE Ts2R fluorescence microscopes. CD spectra were collected using Chirascan Circular Dichroism (CD) Spectrometers. UV-vis absorption spectra were obtained under a Shimadzu UV-2600 spectrophotometer (medium scanning rate and quartz cuvettes of 2 cm path length). Photoluminescence spectra were recorded on an Edinburgh FS5 Spectrofluorometer. Absolute quantum yield was collected using an integrating sphere on a Hamamatsu Quantum Yield Spectrometer C11347 Quantaurus. All digital photographs were recorded on a Canon EOS 7D camera. A Leica SP8 confocal microscope was used for cell imaging.

### Preparation Procedures

Similar to our previous process²⁹, S. chrestomyceticus BCC24770 was cultured in ten 2.5 L Erlenmeyer flasks containing 1 L of GYM (4 g/L glucose, 4 g/L yeast extract, and 10 g/L malt extract; pH 7.2-7.4) and around 100 glass beads (3 mm in diameter) at 30° C. With agitation of 180 rpm for 8 days, the bacterial culture broth was extracted with an equal volume of ethyl acetate three times to obtain 2.1 g of crude extract, followed by isolation through column chromatography by using reverse-phase silica gel (Phenomenex, C18-T, 50 μm, 135 Å) eluted with different gradients of acetonitrile-water solution (1:9 to 10:0) to yield different fractions. After rotary evaporation, HMPQ was accumulated in 40% acetonitrile-water fraction. It was found shining with green fluorescence in dried powders and thus further purified through semi-preparation HPLC under 37% acetonitrile-water isocratic flow, 3 mL/min, with 0.05% trifluoroacetic acid in the mobile phases. Around 15 mg of HMPQ was obtained from 10 L of culture liquids.

### Structural Elucidation

HMPQ with high purity was obtained as a white amorphous powder. The high-resolution mass spectrometer exhibited the [M-H]-peak at m/z 323.1037, indicating the molecular formula of C₁₈H₁₆N₂O₄ with a degree of unsaturation of 12. All proton and carbon signals were detected using DMSO-d₆ for NMR tests. Analyses of ¹H, ¹³C, COSY, HSQC, and HMBC spectra for HMPQ revealed a methoxyl group, a methyl group, and a methylene group connected with a carbonyl carbon to form the propionyl group. Six aromatic protons were on two nonadjacent ring systems, two of them were adjacent protons on a benzene ring and the other four were continuously linked on a quinazolinone with confirmed COSY signals. For the two active protons, one of them was hydroxyl group connected with a benzene ring to form a 1-(2-hydroxy-4-methoxyphenyl)propan-1-one structure; and the other active proton was an amide proton on quinazolinone. The HMBC signals from the amide proton to the C14 of the 1-(2-hydroxy-4-methoxyphenyl)propan-1-one structure and the aromatic proton H12 to C2 connected the two moieties together.

### X-Ray Crystallography Analysis of HMPQ

The sample was dissolved in a glass vial with a sole methanol solvent and sealed with parafilm to evaporate slowly. After one week, two single-crystal samples were obtained, namely, a colourless plate-like crystal in the bottom and a yellow needle-like crystal on the vial membrane. Crystal samples with approximate dimensions of 0.13×0.12×0.03 mm³ and 0.3×0.02×0.02 mm³ were collected, immersed in Paratone-N, and mounted with MiTeGen™ cryo-loops. HRMS confirmed that they had the same molecular weight. Data were collected at 100 K using a Rigaku OD Supernova Diffractometer with Cu-Kα radiation (λ=1.54184 Å). Using Olex2³⁰, the structures were solved with the SHELXT³¹ structure solution program by using intrinsic phasing and refined with the olex2.refine³² refinement package using Gauss-Newton minimization. The resulting monoclinic crystal structures with P2₁/c and P2₁/n space groups were refined successfully to a conventional R1=5.32% and 8.12%, respectively. The CIFs were deposited onto CCDC CSD with codes #2311188 and #2311189.

### Cell Culturing and Cytotoxicity Assay

HEK293 and HeLa cells were purchased from ATCC, and HT22 cells were purchased from Sigma-Aldrich. They were cultured in Dulbecco's modified Eagle's medium (Thermo Fisher Scientific, 11965092) with 10% fetal bovine serum (Thermo Fisher Scientific, 26140079) and 1% penicillin-streptomycin at 37° C. in a humidified incubator with 5% CO₂. After 1 day of cell culture, cells were seeded onto 96-well plates with a density of 5000 cells in each well and cultured for another 24 h. Then, HMPQ with different concentrations was added and treated for 24 h. After incubating with 20 μL of thiazolyl blue tetrazolium bromide (5 mg/mL in PBS buffer; Sigma-Aldrich) for 4 h, supernatants were removed, and 100 μL of DMSO was added to dissolve formazan. After shaking for 5 min in darkness, a Multiskan FC microplate was used to measure absorbance at 570 nm.

### G4 DNA Sample Preparation

Single DNA nucleotides were purchased from Integrated DNA Technologies. The DNA sample at 100 μM (single strands) was then re-annealed by heating to 95° C. for 15 min, followed by slow cooling to room temperature overnight in an annealing buffer of 70 mM KCl and 20 mM potassium phosphate (pH 7.0). The final NMR samples contained 0.1 mM DNA or RNA in 20 mM potassium phosphate buffer (pH 7.0) and 70 mM KCl.

### One-Dimensional (1D) ¹H-NMR Titration Experiments

To verify whether the compounds interacted with G4 DNA, we performed NMR titration experiments by running 1D ¹H NMR spectra at 25° C. All compounds were dissolved in isotope-labelled d₆-DMSO (from Sigma-Aldrich) at about 50 mM concentration as stock solutions. To avoid chemical shifts of G4 samples resulting from d₆-DMSO addition, 10 μL of d₆-DMSO was added into 500 μL of 0.1 mM G4 solution in the NMR buffer (20 mM potassium phosphate buffer and 70 mM KCl, pH 7.0, 10% D₂O). Then, a 1D ¹H-NMR spectrum was obtained as a reference. During NMR titration experiments, each compound was added to the G4 sample solution, and the maximum volume of 10 μL of each compound in the d₆-DMSO solution was considered as the final data point.

### CD Spectroscopy Test

CD spectra of G4s with HMPQ were recorded on a Chirascan CD spectrometer at room temperature from 220 nm to 320 nm under liquid mode. A 1 mm-path-length quartz cuvette was used with a 400 μL sample. Each G4 sample was prepared at the same concentration of 15 μM. The compound was mixed with G4s at a molar ratio of 1:10. Each test was scanned three times, and the background of the buffer was subtracted.

### Cell Imaging

HeLa cells were seeded onto a 12-well plate with coverslips at 37° C. and 5% CO₂ for 1 day and incubated with HMPQ at 10 μM overnight. The next day, cells were fixed with 4% paraformaldehyde for 15 min at room temperature and then permeabilized with 0.2% Triton X-100 in PBS for another 10 min. G4-specific antibodies BG4 (Sigma-Aldrich, cat. no. MABE 1126) were added at 1:500 dilution for staining and incubated overnight at 4° C. Cells were washed three times with PBS and then incubated with secondary antibodies (Alexa 647 anti-rabbit for G4) at 1:1000 dilution for 1 h at room temperature. After washing the cells with PBS three times to remove excess secondary, the cells were incubated with DAPI for 5 min and washed with PBS once. The cells on slides were then air dried, mounted onto a Hydromount medium, and observed using a Leica SP8 confocal microscope.

### Live Cells Imaging

HMPQ is dissolved with polymer Pluronic F-127 together with organic solvent tetrahydrofuran (THF) and water, and then the mixture is sonicated²⁴. After removing THF and filtering, synthesized nanoparticles are obtained. The nanoparticles are incubated with live cells overnight. Live cells containing HMPQ nanoparticles are observed using a Leica SP8 confocal microscope.

### Statistical Analysis

All data were obtained from at least three independent preparations. Quantifications were performed in a blinded manner. Statistical analysis was performed with Origin 2018.

### Data Availability

Crystallographic data for the structures (conformer type 1 and type 2) reported have been deposited at the Cambridge Crystallographic Data Centre (CCDC) under deposition numbers CCDC codes #2311188 and #2311189, respectively. These data can be obtained free of charge via the CCDC worldwide website: ccdc.cam.ac.uk/structures/. All data supporting the findings of this research are available within the Article and its Supplementary Information.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Results

### EXAMPLE 1—Discovery of Natural Bacterial Metabolite AIEgen

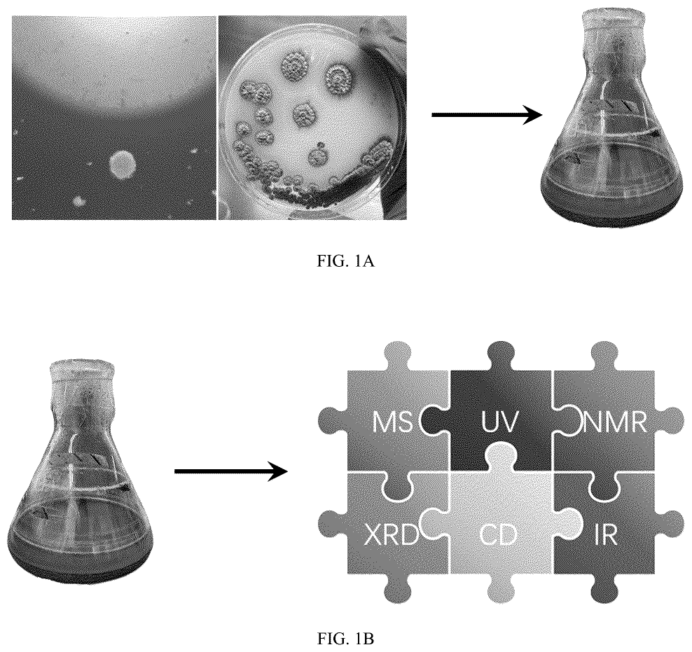

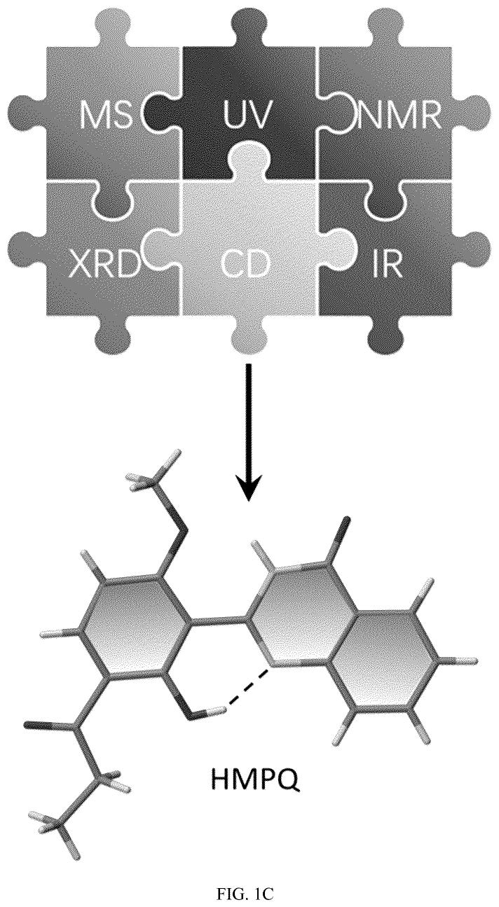

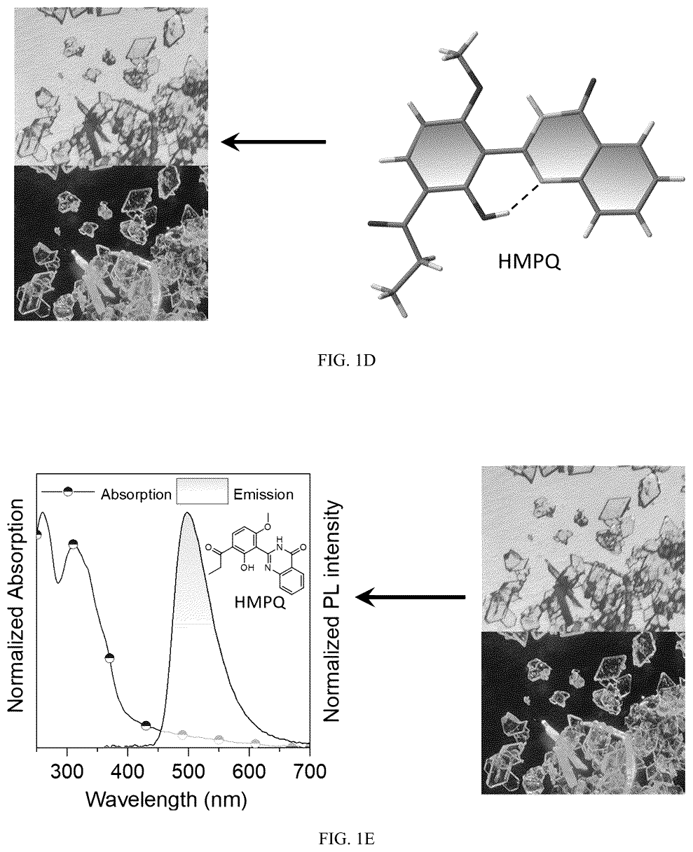

In the pursuit of discovering novel bioactive compounds from marine bacteria, several complex compounds such as chrexanthomycins^{28,29} and albofungins^{33,34} have been successfully isolated through large-scale fermentation in our previous studies (FIG. 1A). This endeavour has resulted in accumulating and identifying previously unknown secondary metabolites, albeit with limited yield. Serendipitously, during HPLC preparation, a novel small molecule was discovered from the marine bacterium Streptomyces chrestomyceticus BCC24770. This molecule exhibited intense green luminescence in a powdered state under ultraviolet (UV) excitation, suggesting its potential as an AIEgen.

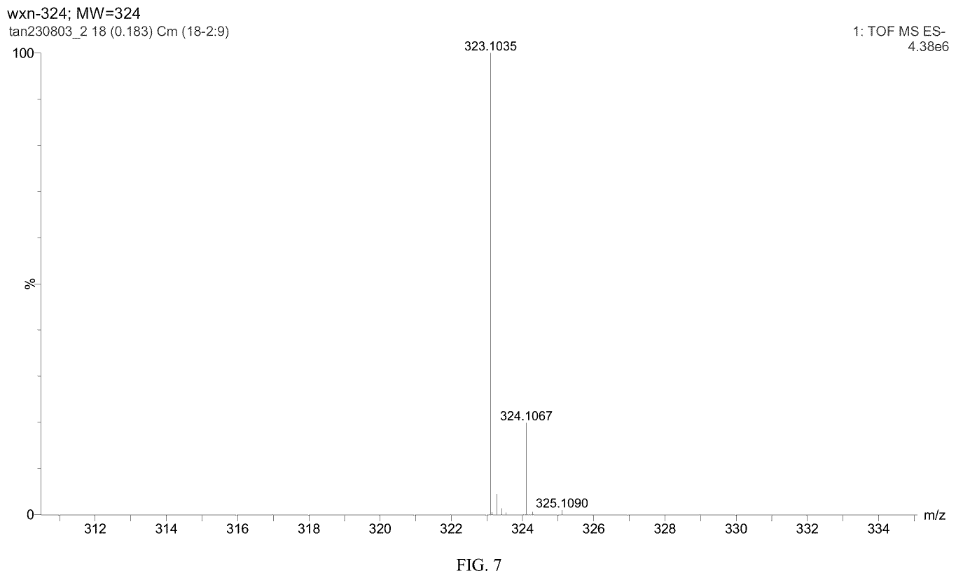

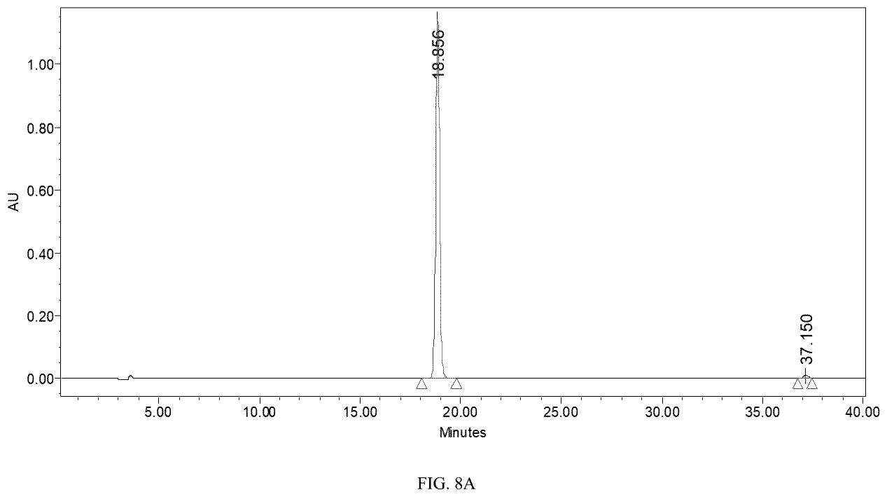

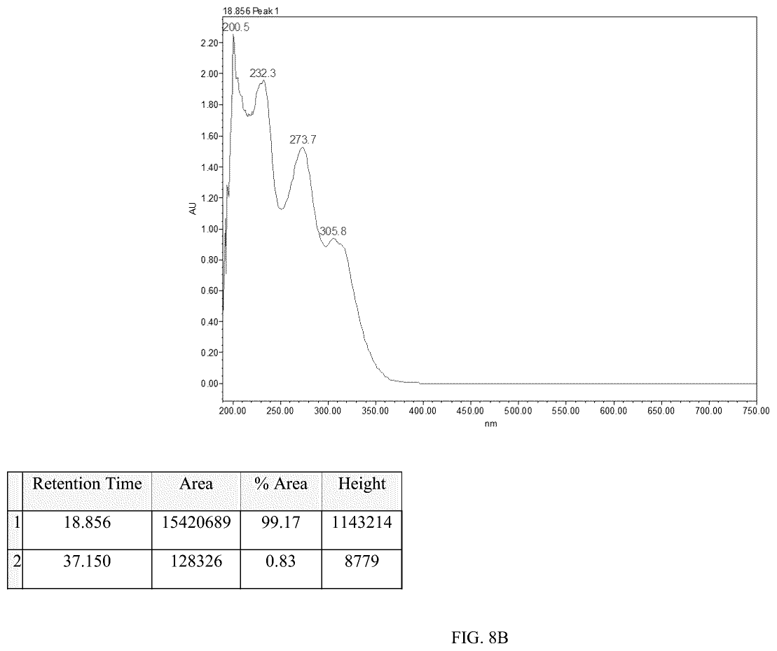

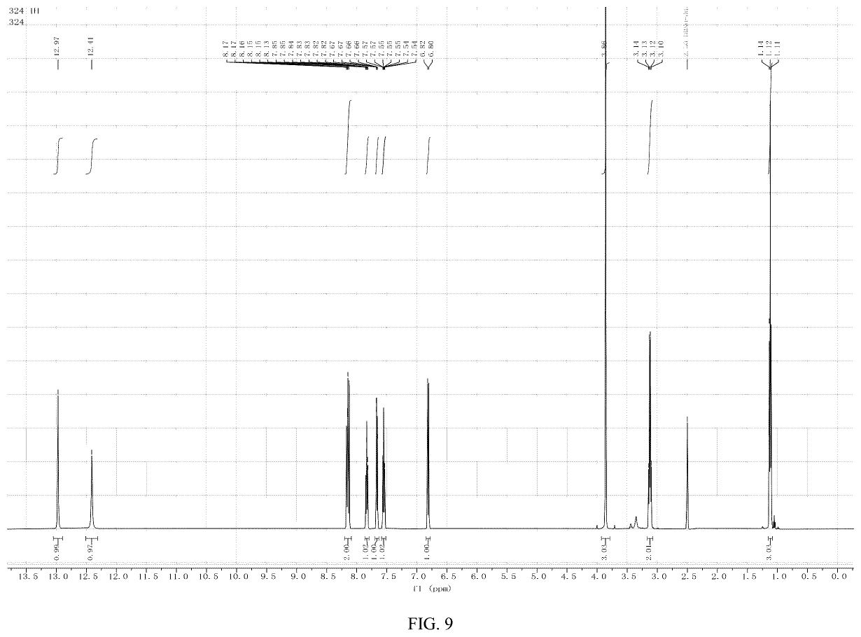

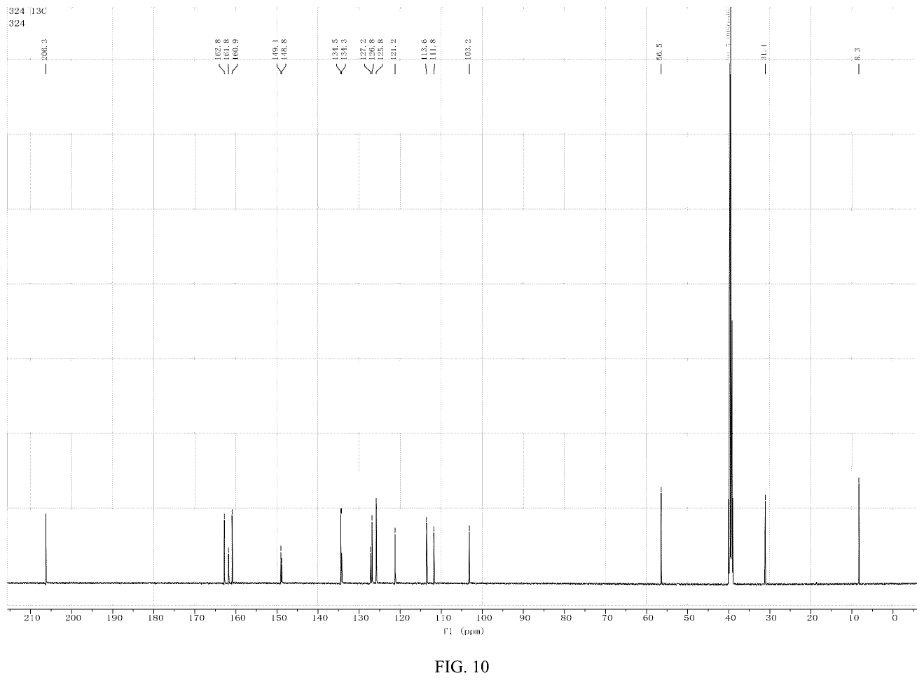

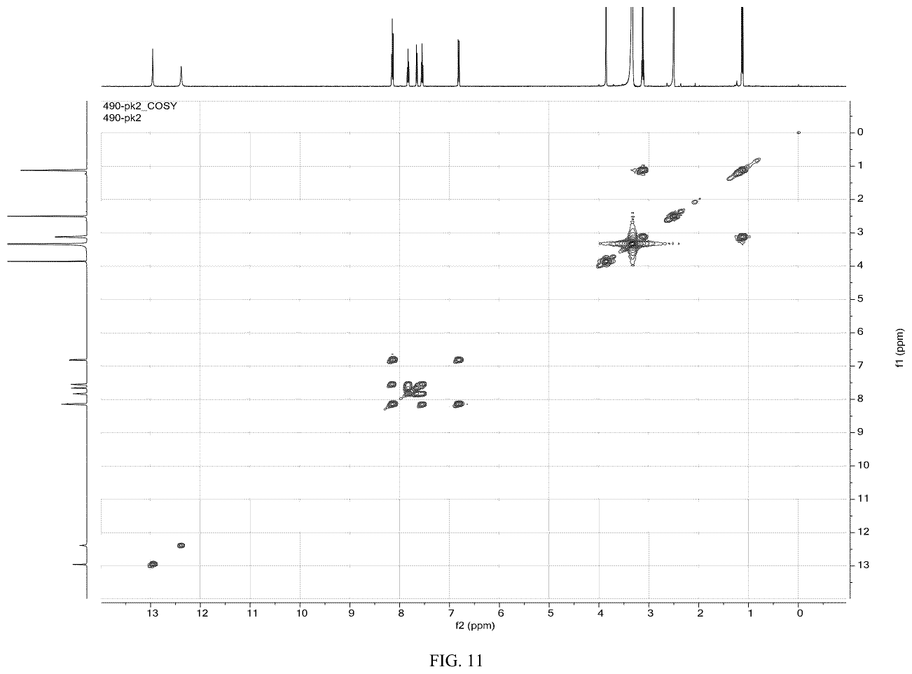

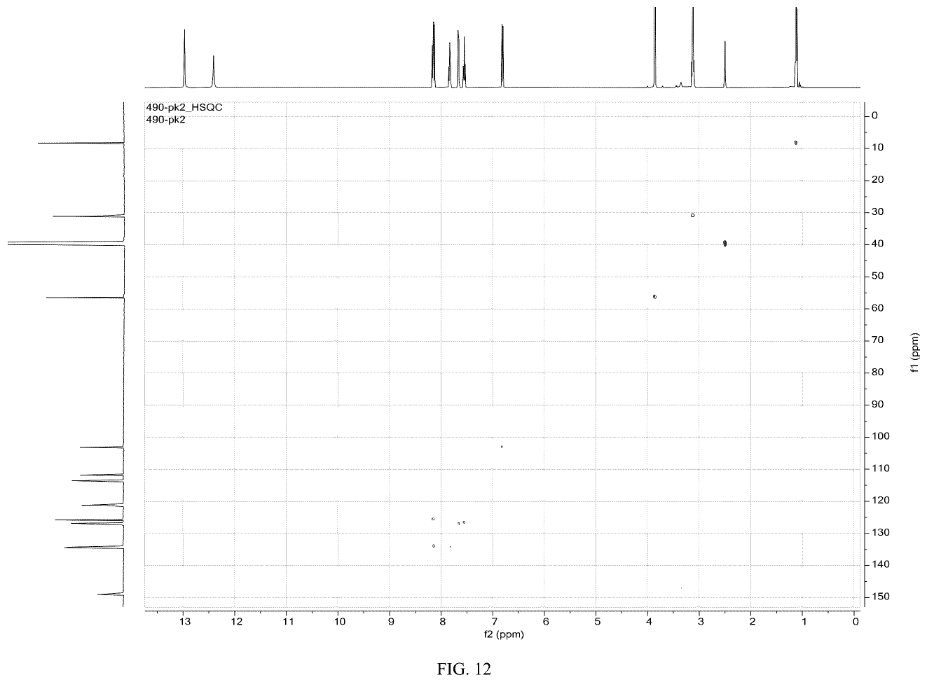

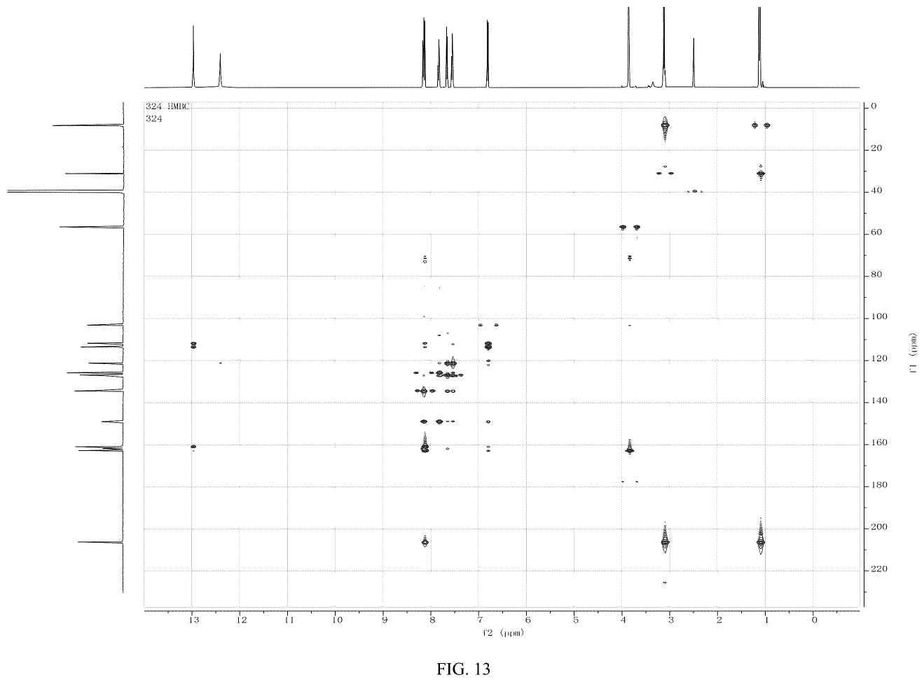

The structure of this compound was established through comprehensive spectrometric analyses (FIG. 1B, including high-resolution mass spectrometry (HRMS), 1D and 2D nuclear magnetic resonance (NMR), and single-crystal X-ray diffraction (SXRD) (FIGS. 7-13, Table 1). The compound was identified as 2-(2-hydroxy-6-methoxy-3-propionylphenyl)quinazolin-4(3H)-one (FIG. 1C). It contained the 4(3H)-quinazolinone scaffold, which is commonly found in plant, fungal, and bacterial metabolites. 4(3H)-Quinazolinones are also well known for their diverse pharmacological activities^{35,36}. As depicted in FIG. 1D, the marine AIEgen HMPQ can be readily crystallised in sufficient quantity. The resulting crystal samples emitted vibrant luminescence under UV irradiation, with a peak emission wavelength at 500 nm (FIG. 1E).

Atom labelling of HMPQ and its ¹H-¹H COSY and key HMBC correlations.

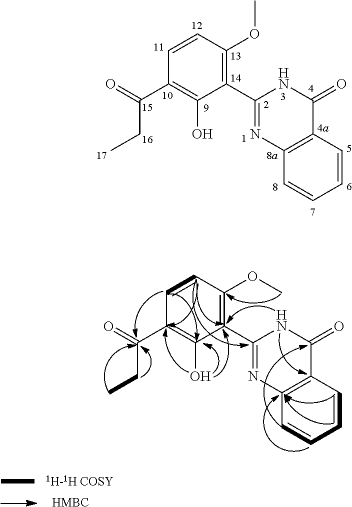

TABLE 1
¹H NMR (500 MHz, DMSO-d₆) and ¹³C NMR (126 MHz) data for HMPQ.

| | | | ¹H—¹H | |
|---|---|---|---|---|
| Position | δ_{C} | δ_{H} (J in Hz) | COSY | HMBC^{a} |
| 2 | 149.1, C | | | |
| 3 | | 12.41, s | | 4a, 14 |
| 4 | 161.8, C | | | |
| 4a | 121.2, C | | | |
| 5 | 125.8, CH | 8.16, dd, (7.9, 1.6) | 6 | 8, 8a |
| 6 | 126.8, CH | 7.55, ddd, (8.1, 7.1, 1.2) | 5, 7 | 4a, 5, 7, 8, 8a |
| 7 | 134.5, CH | 7.83, ddd, (8.5, 7.2, 1.6) | 6, 8 | 4a, 5, 8, 8a |
| 8 | 127.2, CH | 7.66, m | 7 | 4a, 4, 5, 6, 7, 8a, |
| 8a | 148.8, C | | | |
| 9 | 160.9, C | | | |
| 9-OH | | 12.97, s | | 9, 10, 13, 14, 15 |
| 10 | 113.6, C | | | |
| 11 | 134.3, CH | 8.14, d, (9.1) | 12 | 9, 10, 13, 14, 15 |
| 12 | 103.2, CH | 6.81, d, (9.1) | 11 | 2, 9, 10, 13, 14 |
| 13 | 162.8, C | | | |
| 13-OCH₃ | 56.5, CH₃ | 3.86, s | | 13 |
| 14 | 111.8, C | | | |
| 15 | 206.3, C | | | |
| 16 | 31.1, CH₂ | 3.12, q, (7.2) | 17 | 15, 17 |
| 17 | 8.3, CH₃ | 1.13, t, (7.2) | 16 | 15, 16 |

^{a}HMBC correlations are from proton(s) to the indicated carbon(s).

### EXAMPLE 2—Photophysical Characterisations of HMPQ

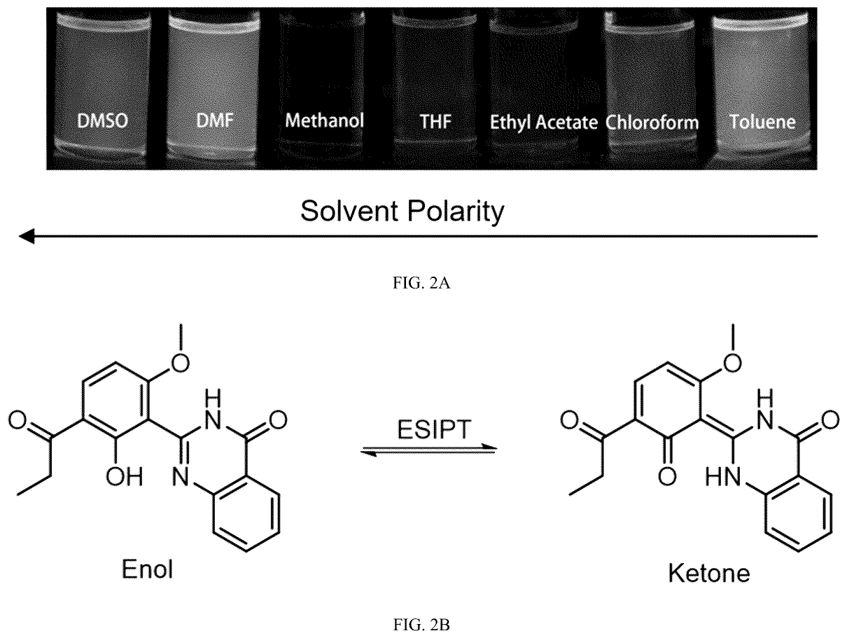

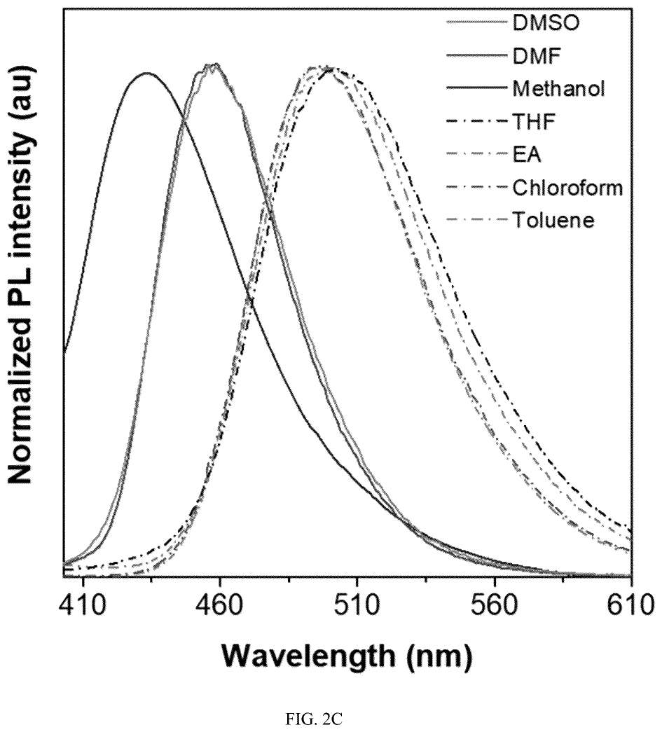

Following the successful isolation of HMPQ in appreciable quantities, systematic characterizations were performed to assess its photophysical properties. ESIPT is a reversible photo-tautomerization process occurring at the excited state, which is facilitated by the intramolecular hydrogen bond. This process has elicited significant research interest owing to its fascinating emission phenomena and potential applications^{25,37,38}. The ESIPT properties of HMPQ rendered its fluorescence emission exquisitely sensitive to the polarity of the surrounding microenvironment (FIG. 2A).

In polar aprotic solvents such as dimethyl sulfoxide and dimethylformamide, the ESIPT tautomerization from enol to keto form was primarily inhibited. The outcome was bright-blue fluorescence that peaked at 457 nm under photoexcitation. As solvent polarity decreased, the ESIPT mechanism switched on, facilitated by less-polar solvents better able to stabilize the intramolecular hydrogen bond in the excited state. This phenomenon manifested as a dramatically redshifted emission to 497 nm in moderately polar solvents like chloroform, culminating in peak green fluorescence in the nonpolar toluene (FIG. 2B). This ESIPT-enabled modulation in emission spanned ˜60 nm from methanol (MeOH) to toluene (FIG. 2C), demonstrating the exceptional solvatochromic range provided through excited-state tautomerization (keto-enol).

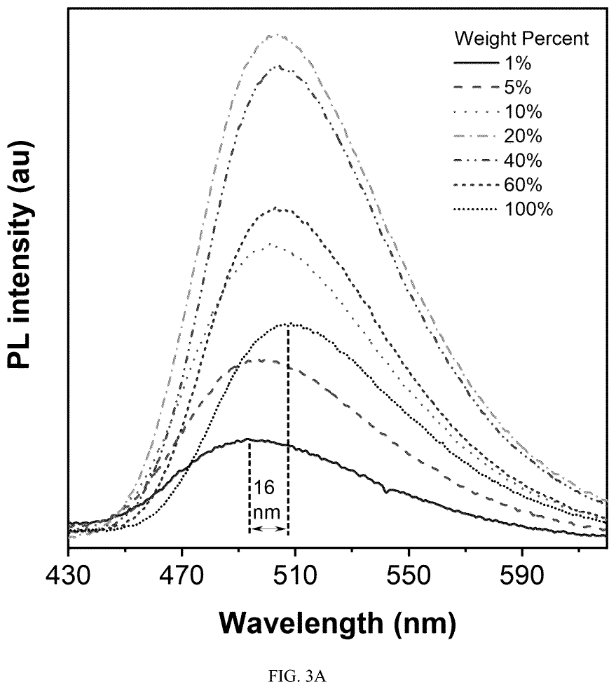

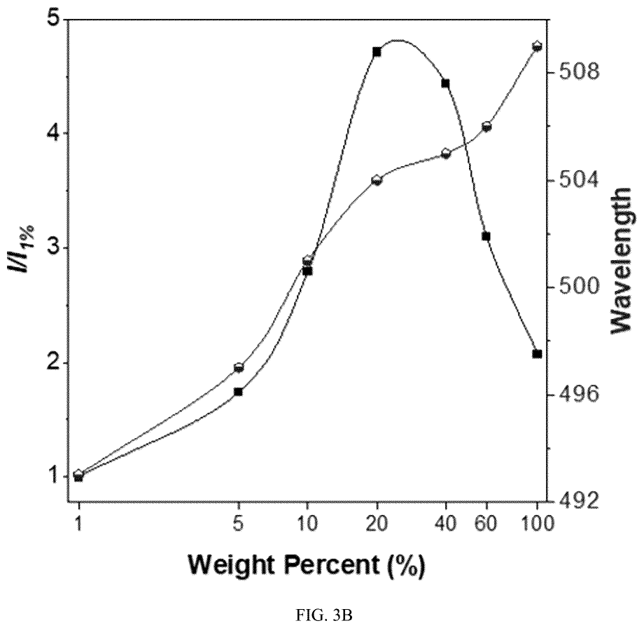

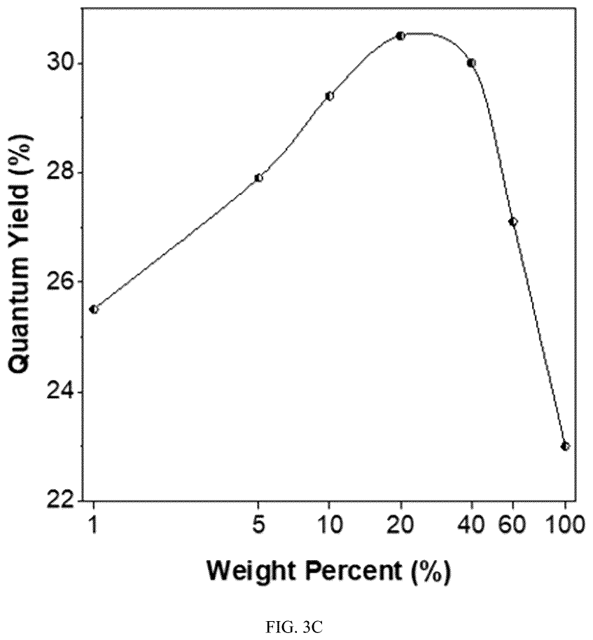

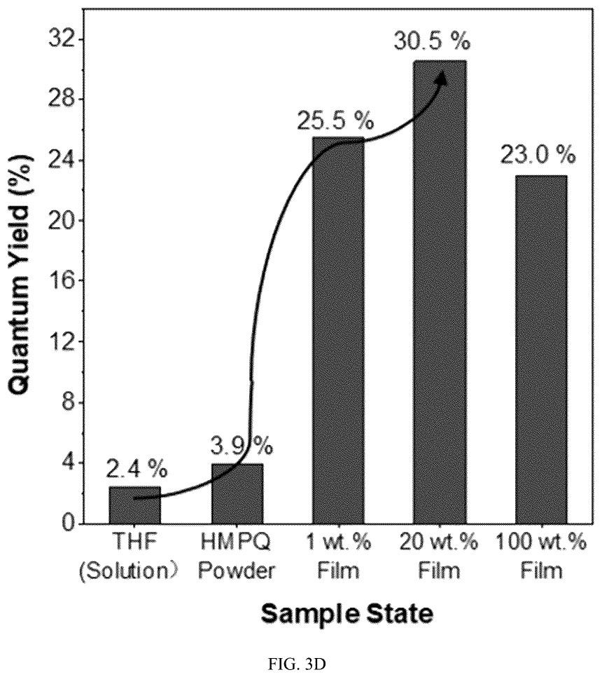

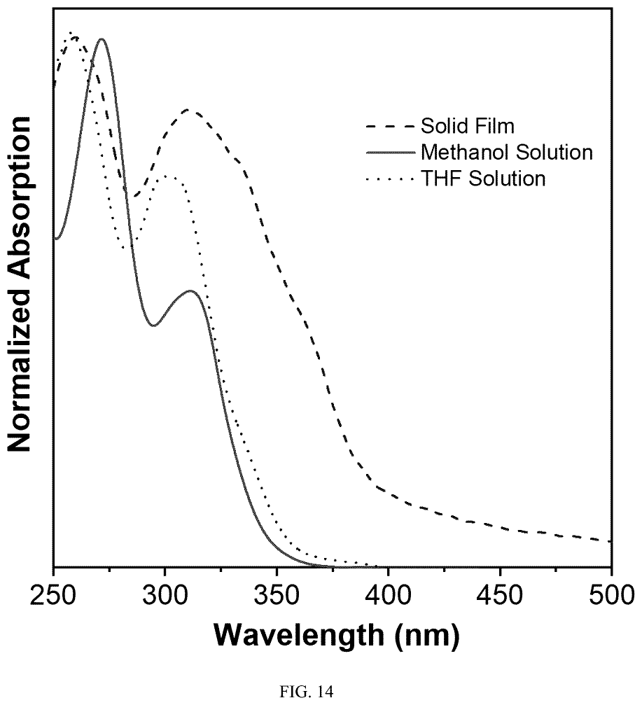

After observing the strong emission of crystalline-state HMPQ, we studied its photophysical properties in a polymer matrix. HMPQ was dissolved and blended with the common polymer poly(methyl methacrylate) (PMMA)³⁹. A series of films was prepared by coating mixtures with varying weight percentages (wt %) of HMPQ onto a quartz substrate. The resulting films, containing different wt % of HMPQ, were analysed to measure their photoluminescence (PL) and quantum yield (QY) (FIGS. 3A-3D and 14, Table 2).

FIGS. 3A and 3B show that PL intensity increased with increased wt % of HMPQ. The intensity peaked at approximately 20 wt %, exhibiting a maximum intensity that was 4.8 times greater than the intensity observed at 1 wt %. The emission maxima did not shift with increased HMPQ concentration up to 20 wt %, but a further increase led to a gradual redshift, suggesting greater π-π interactions. This finding was consistent with the observed redshift between 1 wt % and 100 wt % samples in FIG. 3A. In FIG. 3C the QY of the samples followed the PL intensity change. FIG. 3D shows a distinctly enhanced QY versus solution state (THF solution QY of 2.4%). The 30 wt % film reached a QY of 30.5%. The higher film versus solution QY suggested the AIE nature of this molecule. A distinct enhancement in QY in solid-state emission compared with its solution-state counterpart is illustrated in FIG. 3D. The QYs of the abovementioned samples are summarized in Supplementary Table 2.

TABLE 2
Quantum yield of HMPQ in different states.^{a}

| | | | | Quantum |
|---|---|---|---|---|
| Entry | HMPQ State | [HMPQ] | | Yield (%) |
| 1 | Solid Powder | / | | 3.9 |
| 2^{b} | Film | 1 | wt. % | 25.5 |
| 3^{b} | Film | 5 | wt. % | 27.9 |
| 4^{b} | Film | 10 | wt. % | 29.4 |
| 5^{b} | Film | 20 | wt. % | 30.5 |
| 6^{b} | Film | 40 | wt. % | 30.0 |
| 7^{b} | Film | 60 | wt. % | 27.1 |
| 8^{b} | Film | 100 | wt. % | 23.0 |

| | | | |
|---|---|---|---|
| 9^{b} | In THF Solution | 10⁻⁵ M | 2.4 |
| 10 | In DMSO Solution | 10⁻⁵ M | 6.7 |
| 11 | Crystal Conformer type 1 | / | 17.4 |
| 12 | Crystal Conformer type 2 | / | 2.2 |

^{a}Excitation wavelength: 320 nm.
^{b}Quantum yield of film made by different weight percentages of HMPQ in PMMA mixtures.

### EXAMPLE 3—Polymorphs of HMPQ

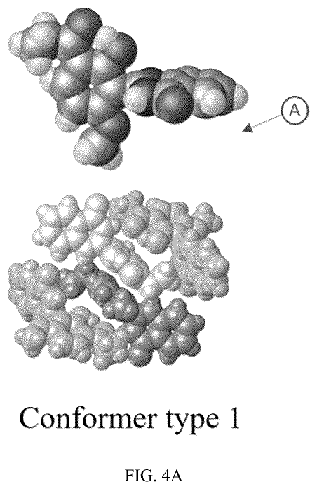

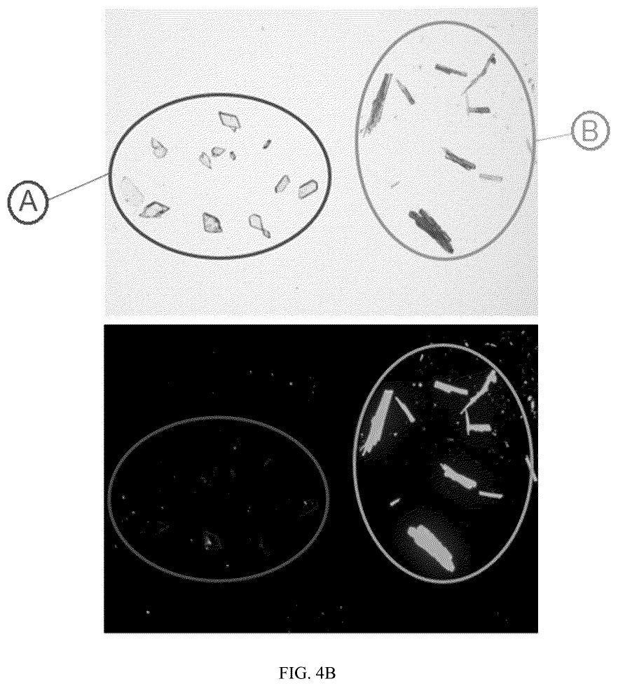

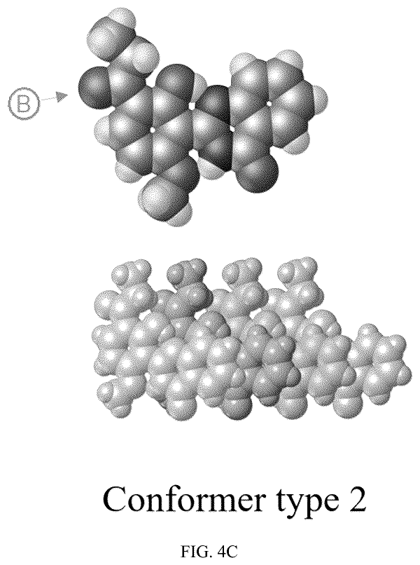

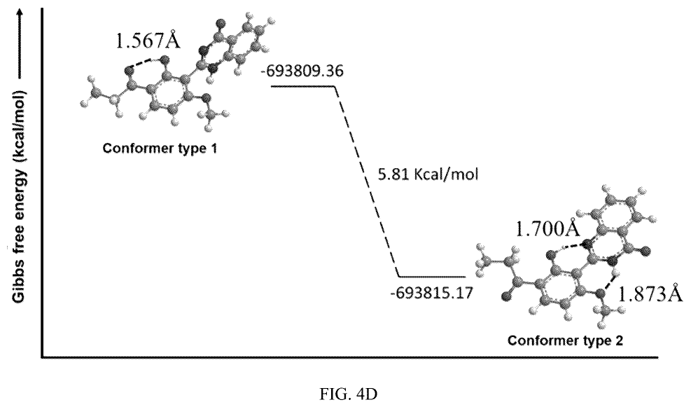

Crystals of HMPQ exhibited strong greenish-blue fluorescence but with a non-uniform pattern upon UV inspection. This phenomenon was caused by the generation of two distinct polymorphic forms, namely, yellow needles as the majority, and a few colourless plates, during HMPQ crystallization. The yellow needle-like crystals displayed a notably higher luminosity than the colourless plates. Crystallographic analysis of the two polymorphic forms showed that the colourless plates displayed a twisted conformation (conformer type 1), whereas the yellow needle possessed a planar structure (conformer type 2)^{29,30,31} (Tables 3 and 4). This structural difference substantially impacted their photophysical characteristics (FIGS. 4A-4C). Specifically, in conformer type 1, the 4(3H)-quinazolinone scaffold within these crystals was oriented perpendicularly to the methoxyphenyl ring. The presence of only one intramolecular hydrogen bond within the methoxyphenyl ring led to a significantly low QY of 2.2% under UV excitation. Conversely, conformer type 2 exhibited the presence of two intramolecular hydrogen bonds connecting the quinazolinone scaffold and the methoxyphenyl ring. As a result, crystal samples of this polymorph emitted vivid fluorescence under UV light, achieving an impressive QY of 17.4%.

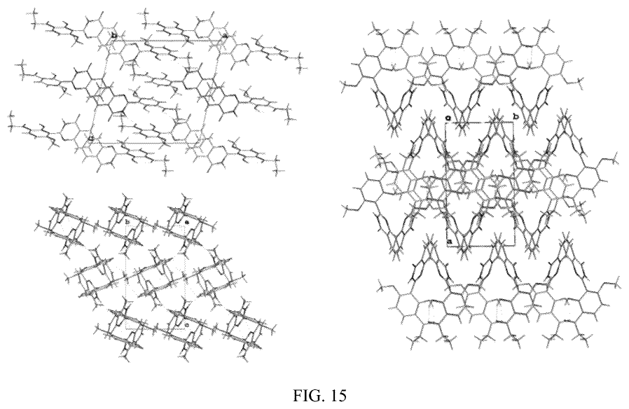

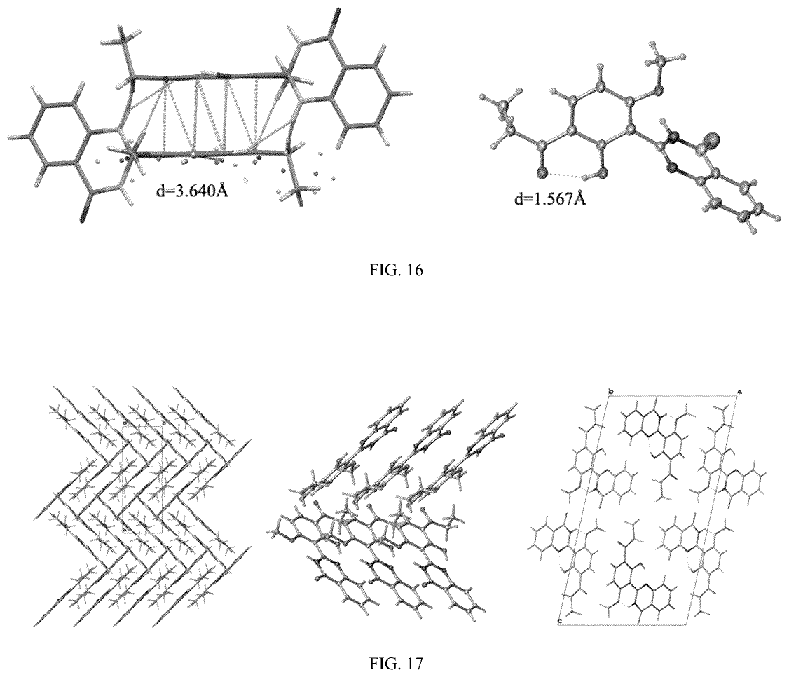

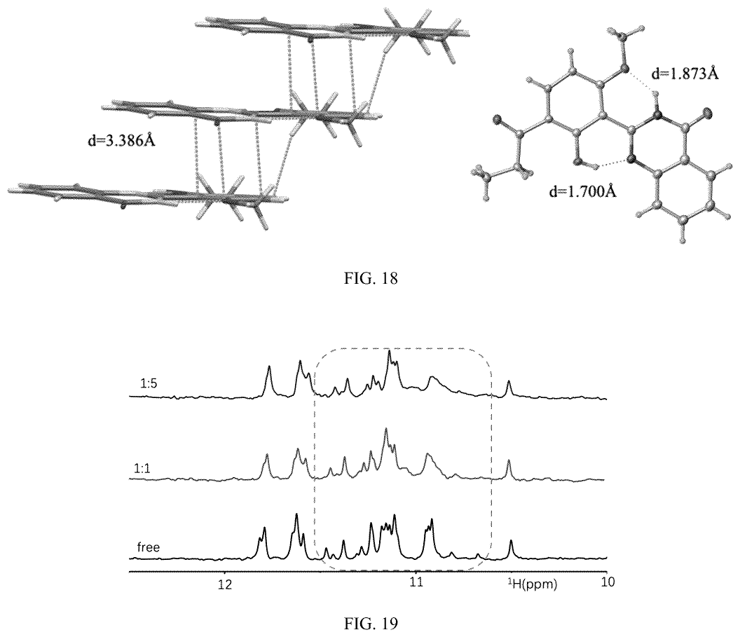

The differences in QY can be ascribed to the disparities among the different conformations and molecular packing within the crystal lattices^{40,41,42}. Conformer type 2 showcased well-organised and densely packed molecular configurations. Conversely, conformer type 1 demonstrated less efficient packing, as evidenced by a larger specific molecular volume and reduced rigidity (FIGS. 15-18). This structural flexibility enabled the rotation of the methoxyphenyl ring in conformer type 1. This stabilizes the excited-state molecular conformation through electron delocalization and resonance, benefiting the excited-state molecular motion. Thus, non-radiative deactivation of the excited state occurred¹². Density functional theory (DFT) calculations illustrated the energy differences between two types of conformers⁴³. When two types of conformers underwent the restrained geometry optimization and frequency analysis at the M062x/TZVP level of theory in the IEFPCM model (methanol)⁴⁴, conformer type 2 possessed a much lower Gibbs free energy (5.81 kcal/mol) than conformer type 1. This result indicated that conformer type 2 was more stable than type 1, offering additional support to our experimental findings (FIG. 4D). Therefore, the connection between the molecular structure and the AIE properties of HMPQ was readily established. Key contributors to the modulation of QYs in different states included conformations, intramolecular hydrogen bonds, molecular stacking, and packing efficiency.

TABLE 3
Crystal data and structure refinement
for ye67CuLT_auto (conformer type 1).

| Identification code | ye67CuLT_auto | |
|---|---|---|
| Empirical formula | C₁₈H₁₆N₂O₄ | |
| Formula weight | 324.33 | |
| Temperature/K | 100.01 | (10) |
| Crystal system | monoclinic | |
| Space group | P2₁/c | |
| a/Å | 14.5023 | (13) |
| b/Å | 7.7430 | (6) |
| c/Å | 13.8019 | (12) |
| α/° | 90 | |
| β/° | 102.790 | (9) |
| γ/° | 90 | |
| Volume/Å³ | 1511.4 | (2) |

| | |
|---|---|
| Z | 4 |
| ρ_{calc}g/cm³ | 1.425 |
| μ/mm⁻¹ | 0.844 |
| F(000) | 680.0 |
| Crystal size/mm³ | 0.13 × 0.12 × 0.03 |
| Radiation | Cu Kα (λ = 1.54184) |
| 2Θ range for data collection/° | 6.25 to 155.954 |
| Index ranges | −17 ≤ h ≤ 18, −7 ≤ k ≤ 9, −11 ≤ |
| | l ≤ 16 |
| Reflections collected | 9224 |
| Independent reflections | 3114 [Rᵢₙₜ = 0.0421, R_{sigma} = 0.0399] |
| Data/restraints/parameters | 3114/180/340 |
| Goodness-of-fit on F² | 1.064 |
| Final R indexes [I >= 2σ (I)] | R₁ = 0.0532, wR₂ = 0.1412 |
| Final R indexes [all data] | R₁ = 0.0646, wR₂ = 0.1493 |
| Largest diff. peak/hole/e Å⁻³ | 0.25/−0.22 |

TABLE 4
Crystal data and structure refinement
for YE72-1_auto (conformer type 2).

| Identification code | YE72-1_auto | |
|---|---|---|
| Empirical formula | C₁₈H₁₆N₂O₄ | |
| Formula weight | 324.33 | |
| Temperature/K | 100.00 | (10) |
| Crystal system | monoclinic | |
| Space group | P2₁/n | |
| a/Å | 13.2012 | (7) |
| b/Å | 4.7868 | (2) |
| c/Å | 24.1402 | (12) |
| α/° | 90 | |
| β/° | 102.567 | (5) |
| γ/° | 90 | |
| Volume/Å³ | 1488.91 | (13) |

| | |
|---|---|
| Z | 4 |
| ρ_{calc}g/cm³ | 1.447 |
| μ/mm⁻¹ | 0.857 |
| F(000) | 680.0 |
| Crystal size/mm³ | 0.3 × 0.02 × 0.02 |
| Radiation | Cu Kα (λ = 1.54184) |
| 2Θ range for data collection/° | 7.066 to 152.528 |
| Index ranges | −14 ≤ h ≤16, −5 ≤ k ≤ 5, −30 ≤ l ≤ 29 |
| Reflections collected | 9242 |
| Independent reflections | 2904 [Rᵢₙₜ = 0.0405, R_{sigma} = 0.0305] |
| Data/restraints/parameters | 2904/0/220 |
| Goodness-of-fit on F² | 1.048 |
| Final R indexes [I >= 2σ (I)] | R₁ = 0.0812, wR₂ = 0.2303 |
| Final R indexes [all data] | R₁ = 0.0936, wR₂ = 0.2375 |
| Largest diff. peak/hole/e Å⁻³ | 0.32/−0.37 |

### EXAMPLE 4—HMPQ as G4 Beacon

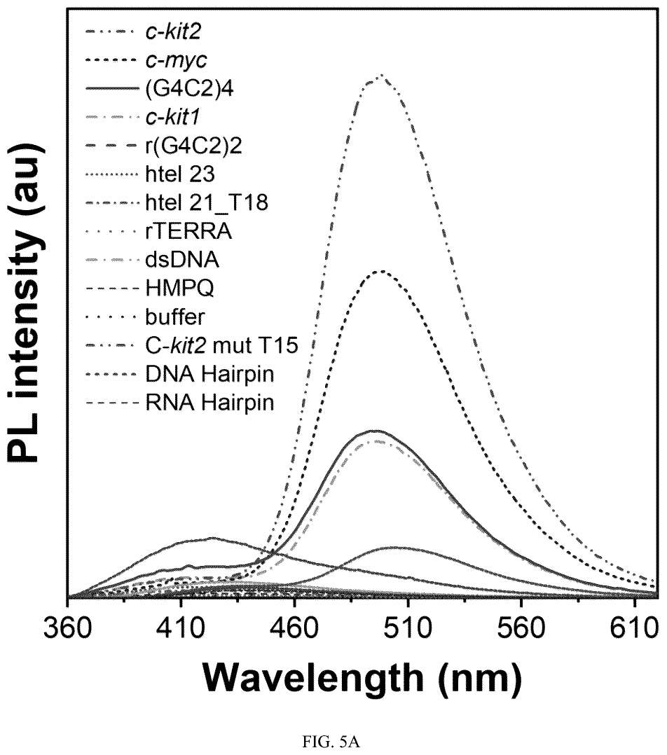

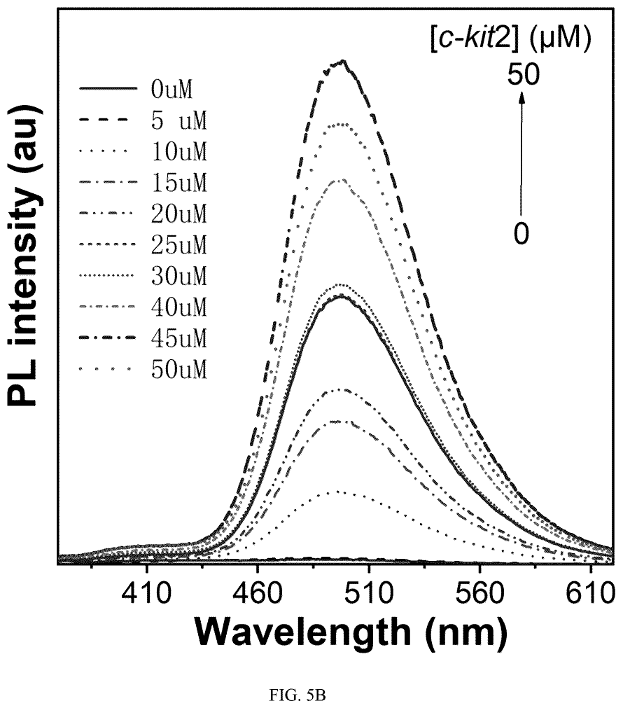

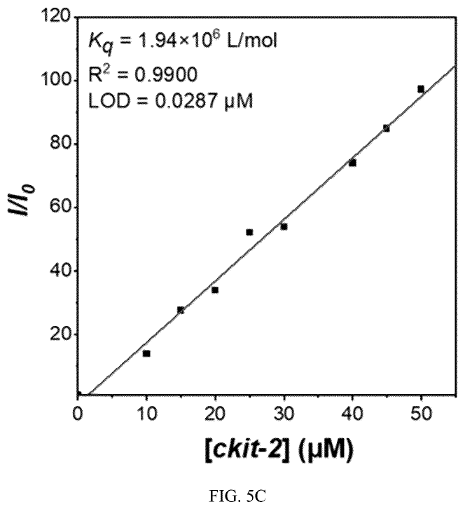

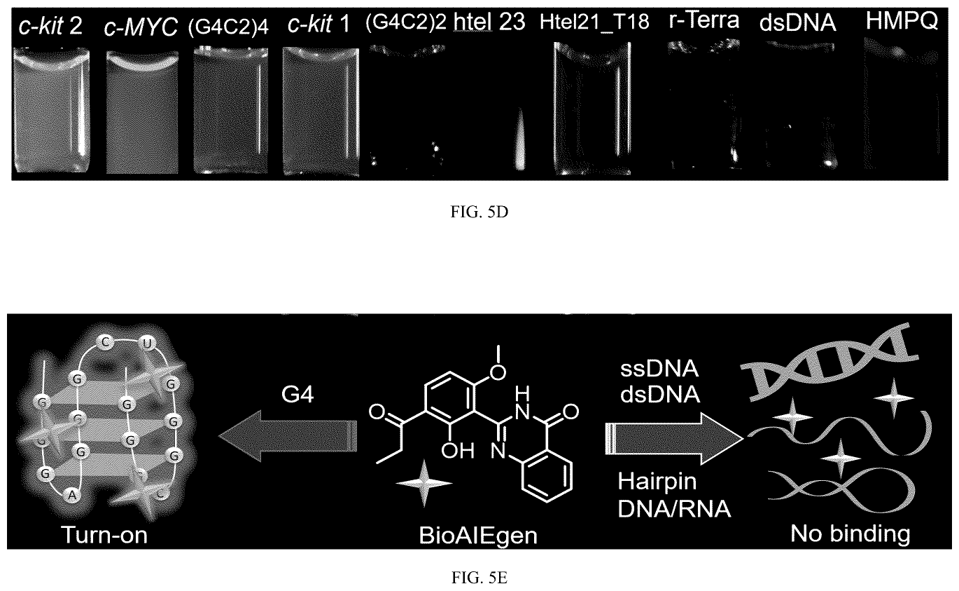

The intriguing conformational change observed in HMPQ led to exceptional emission performance, prompting us to explore its application as a fluorescent probe. The inherent two-planar aromatic rings of HMPQ offer the potential for interaction with the guanine tetrad or conjugation with side chains of novel nucleic acid secondary structures, G4s^{45,46}. To evaluate the binding affinity of HMPQ, a series of photoluminescence tests was conducted using various G4 samples introduced with HMPQ (Table 5). After mixing and filtration, minimal emission was observed in the background solutions containing solely G4 or HMPQ, as depicted in FIGS. 5A and 5D. Conversely, adding HMPQ to specific G4s elicited a remarkably enhanced emission response. The corresponding PL spectra demonstrated that the binding of HMPQ to specific G4s led to a new redshift centred at 497 nm.

Among the repertoire of tested G4s, the most conspicuous “turn-on” effect was discerned in c-kit2 (SEQ ID NO: 3) upon its interaction with HMPQ; the fluorescence at 497 nm was significantly enhanced by up to around 300-fold. Notably, the c-kit2 sequence derived from the KIT proto-oncogene has been proven to regulate cancer pathogenesis^{47,48}. To delve deeper into the sensitivity of HMPQ as a fluorescent G4 probe, the PL spectra of HMPQ (0.15 mM) were obtained with varying concentrations of c-kit2, as illustrated in FIG. 5B. A distinctive and progressive increase in PL intensity was observed with increased c-kit2 concentration from 0 μM to 50 μM. The Stern-Volmer plot in FIG. 5C demonstrates more details on the fluorescence turn-on process, from which the Stern-Volmer constant m was calculated as 1.94×10⁶ M⁻¹. According to the equation for limit of detection (LOD), LOD=3 SB/m (SB=the standard deviation of the repeated 10 blank measurements, and m=the slope of the relative intensity over HMPQ concentration), it was calculated as 28.7 nM^{49,50}. This outcome highlighted the outstanding sensitivity of HMPQ as a specific G4 probe. The graphical representation of HMPQ in FIG. 5E elucidates its capability of selectively recognizing and illuminating G4s, thereby signifying its tremendous potential in facilitating the detection and detailed study of G4 structures.

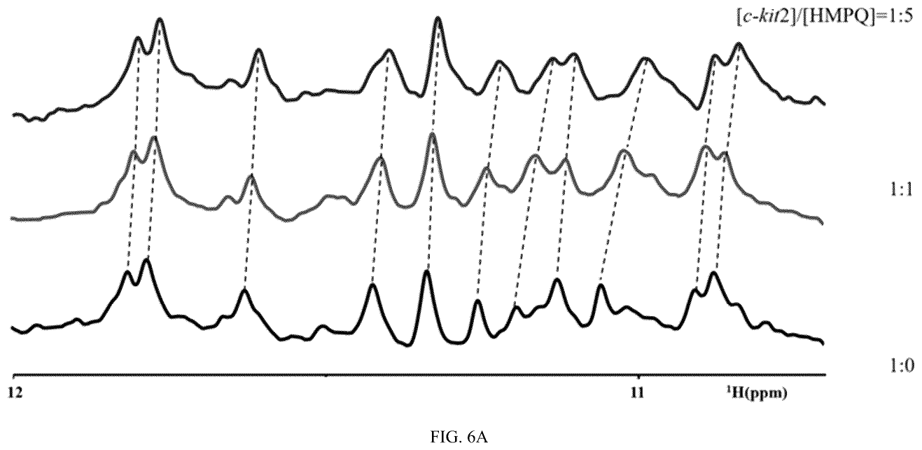

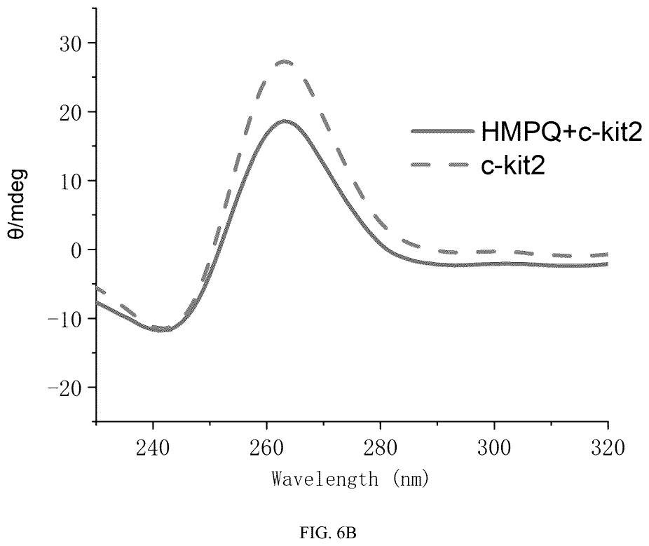

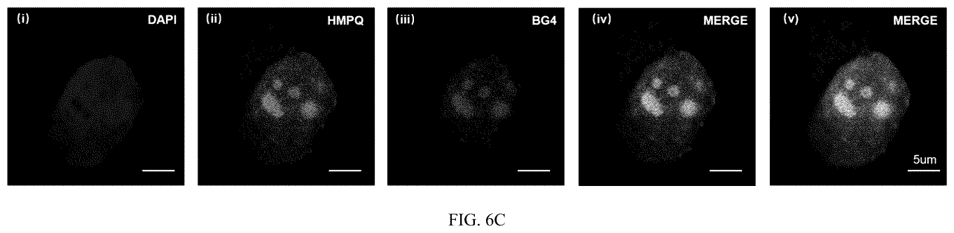

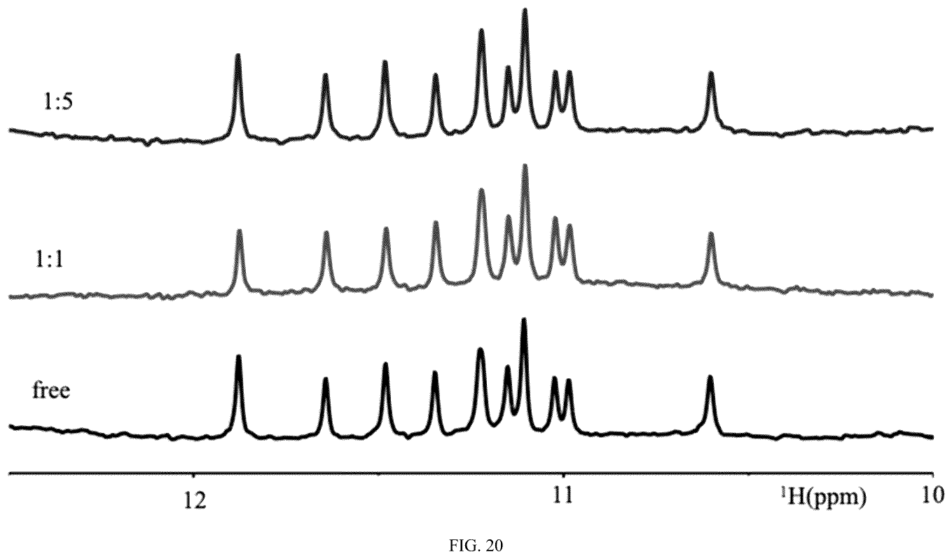

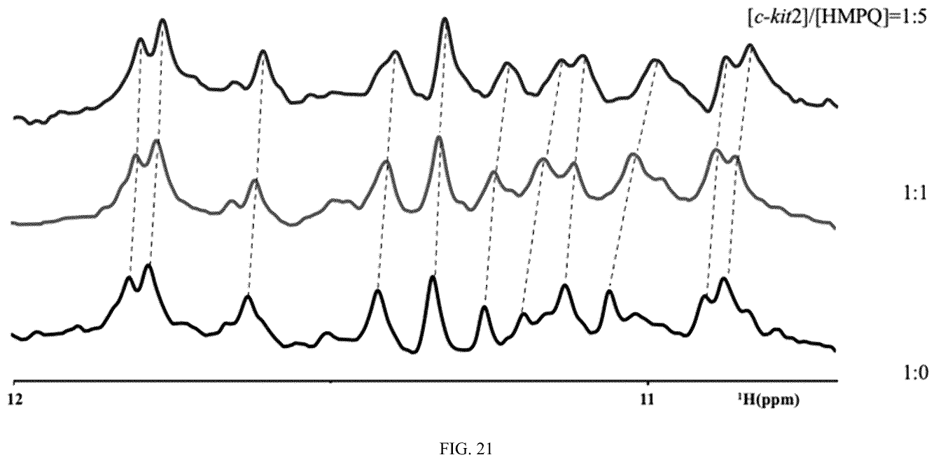

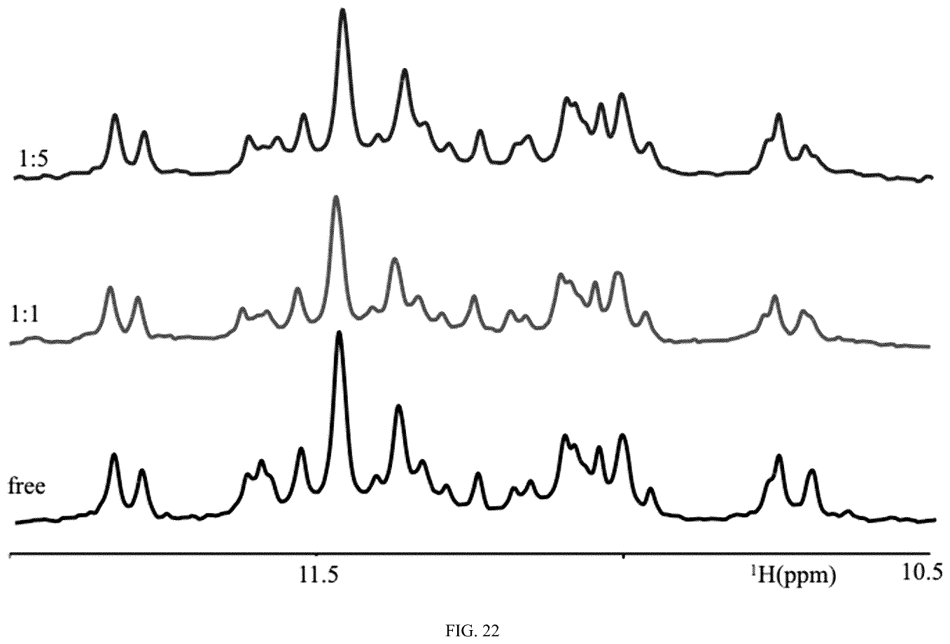

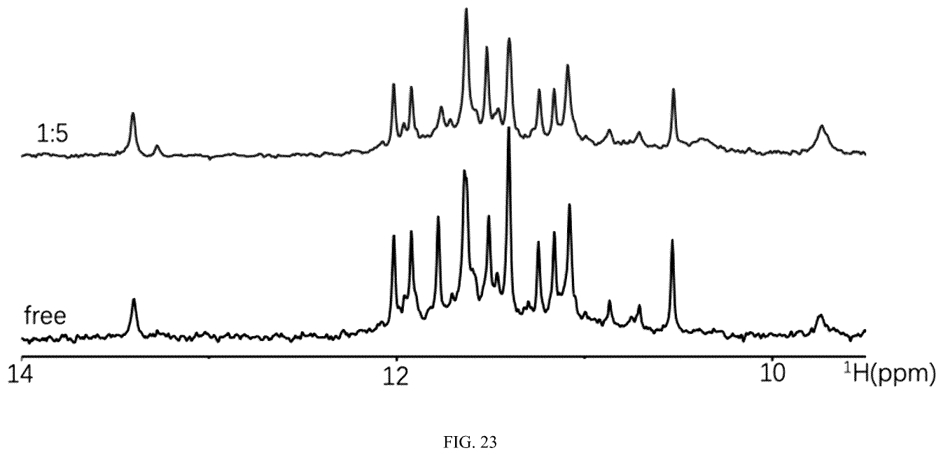

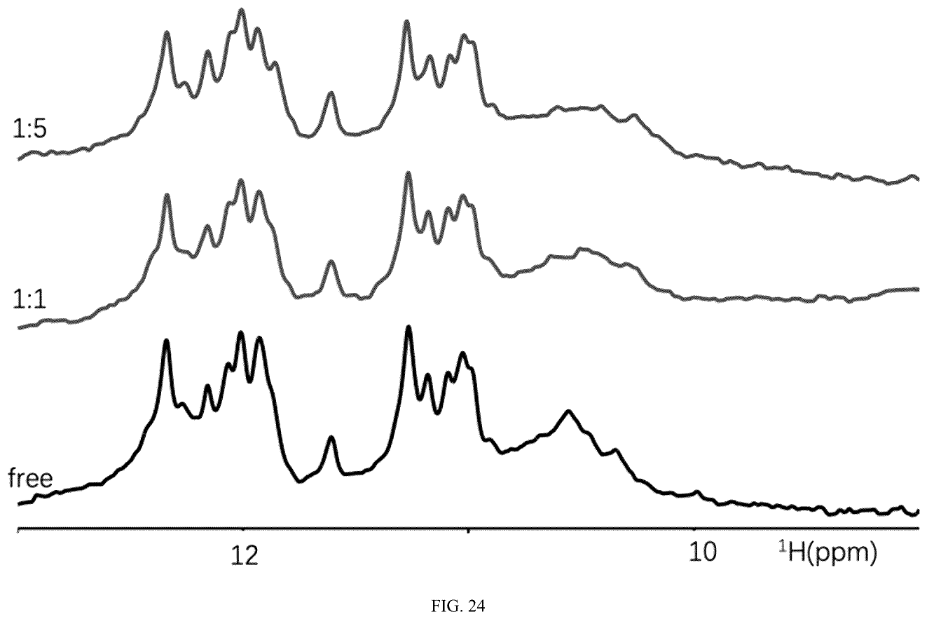

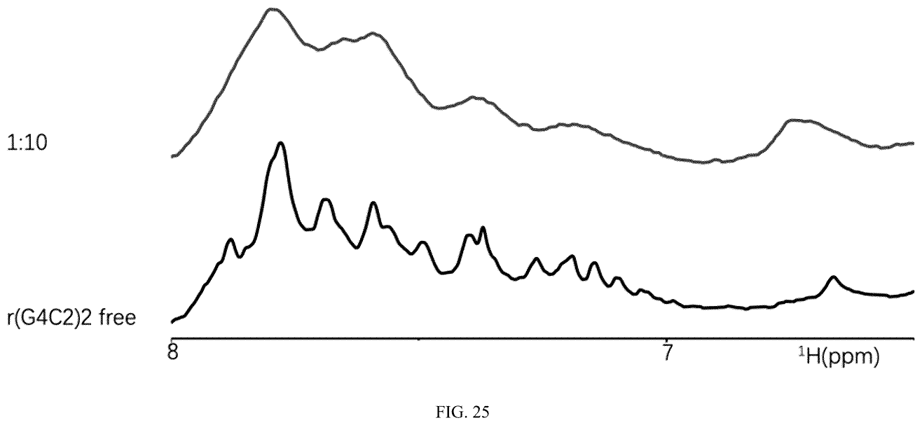

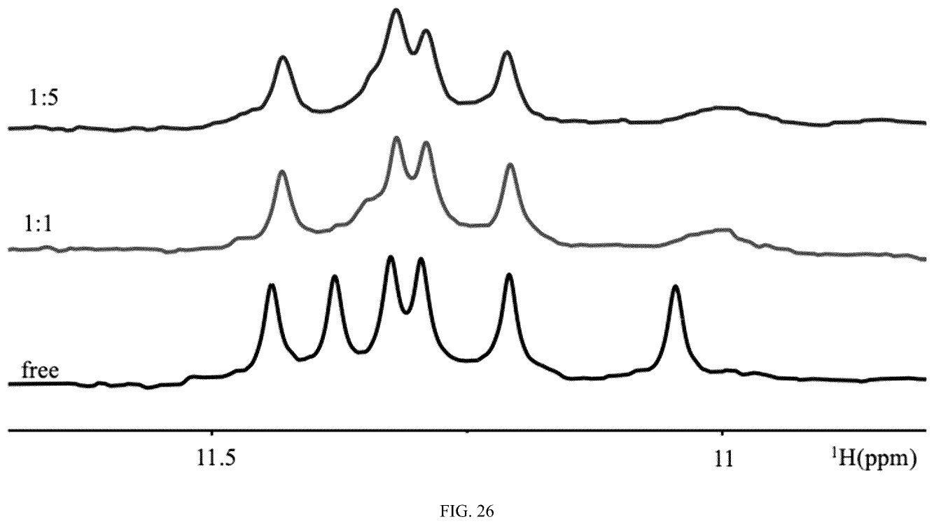

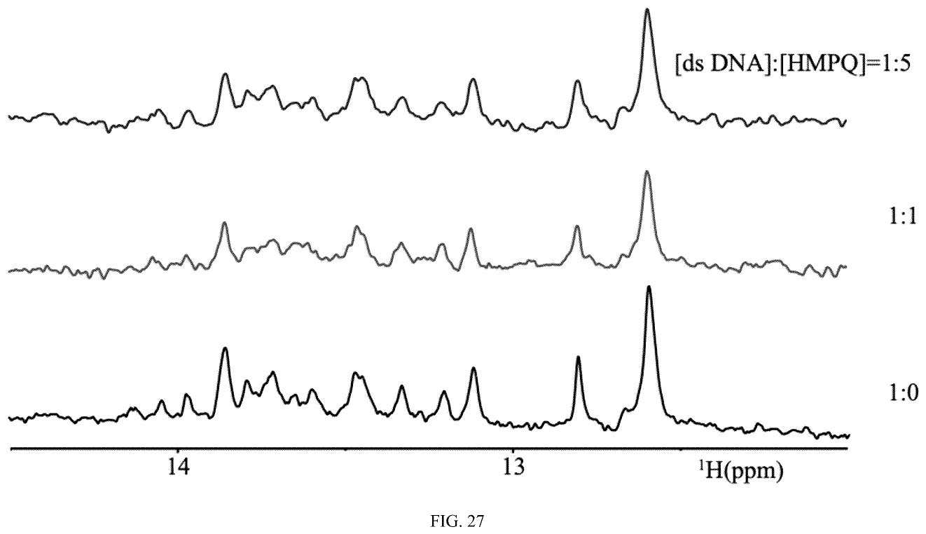

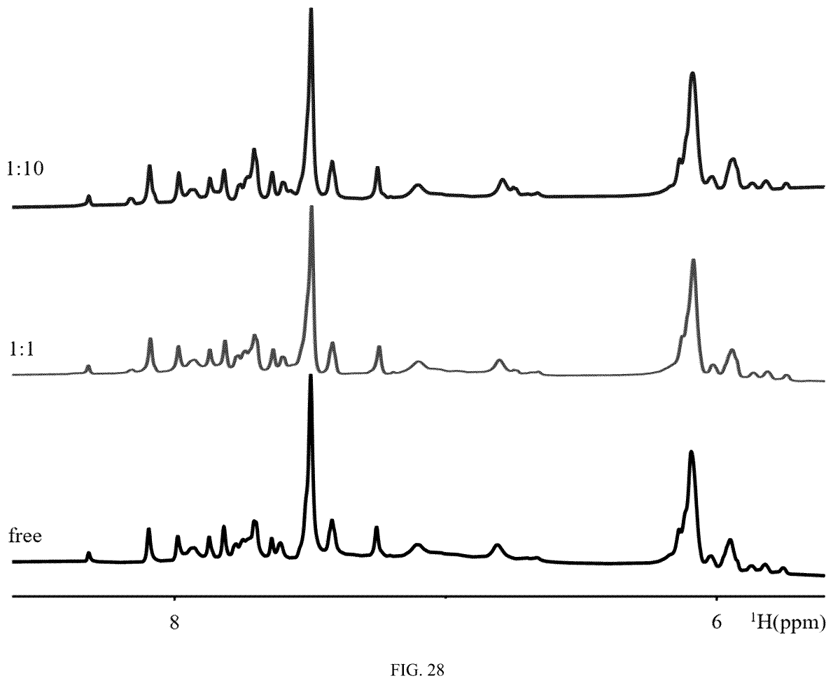

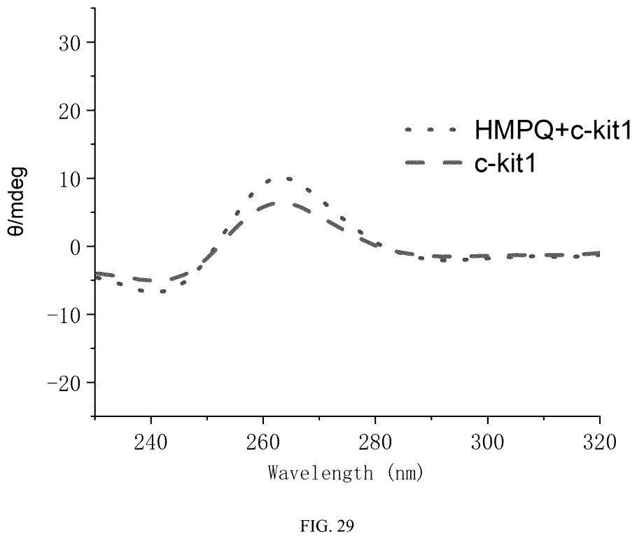

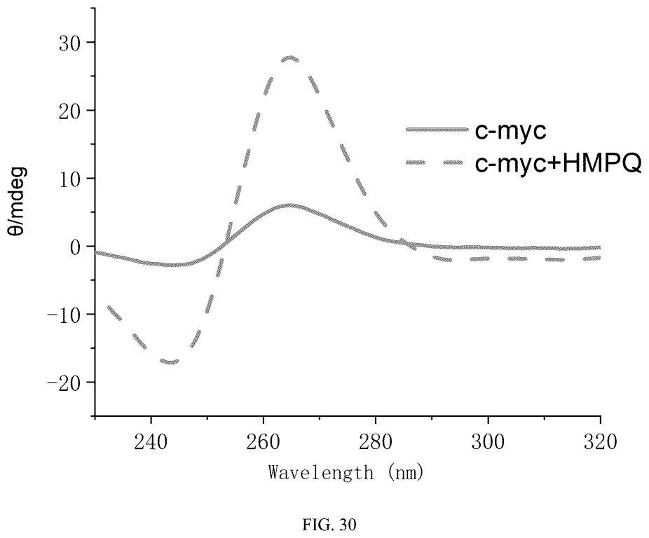

In addition to fluorescence spectroscopy tests, we performed NMR titration as another prevalent method for assessing binding affinity. NMR titration relied on the perturbation of chemical shifts in G4 imino protons upon binding with small molecules⁵¹. Our NMR titration results were consistent with the PL ones (FIGS. 6A, and 19-28), indicating that the binding of the compound illuminated the G4 structure. Moreover, the circular dichroism (CD) spectrum affirmed its non-disruptive nature towards the original conformation of G4s (FIGS. 6B, 29 and 30)

HMPQ demonstrated no cytotoxicity towards human cell lines, including HEK293 and HeLa cells, as well as immortalized mouse hippocampal neuronal cells HT22, even at 100 μM concentration. Such an exceptionally high biosafety profile encouraged us to explore its potential for the precise visualization of subcellular structures and the spatial distribution of G4s within cells. Using BG4, a well-established G4 structure-specific antibody, as a control, HMPQ efficiently penetrated the cell nucleus. This comparison with BG4 generated very intriguing results, as shown in FIG. 6C. HMPQ displayed an impressive overlap coefficient of 94.3% with BG4. This finding indicated that HMPQ and BG4 shared a substantial degree of co-localization, thereby effectively mapping the presence of G4 structures within the cells.

TABLE 5
Oligonucleotides used in this work.

| | | | |
|---|---|---|---|
| | | SEQ ID | |
| Name | Sequences (5′-3′) | NO. | Topology |

DNA G4

| | | | |
|---|---|---|---|
| c-myc | TGA GGG TGG GTA GGG TGG GTA A | 1 | Parallel |
| c-kit1 | AGG GAG GGC GCT GGG AGG AGG G | 2 | Parallel |
| c-kit2 | CGG GCG GGC GCG AGG GAG GGG | 3 | Parallel |
| hte123 | TAG GGT TAG GGT TAG GGT TGG GG | 4 | Hybrid |
| LTR-III | GGG AGG CGT GGC CTG GGC GGG ACT GGG G | 5 | Hybrid |
| htel21_T18 | GGG TTA GGG TTA GGG TTT GGG | 6 | Antiparallel |
| (G4C2)4 | GGG GCC GGG GCC GGG GCC GGG GCC | 7 | Antiparallel |

RNA G4

| | | | |
|---|---|---|---|
| r(G4C2)2 | GGG GCC GGG GCC | 8 | Parallel |
| rTerra | UAG GGU UAG GGU | 9 | Parallel |

ds DNA

| | | | |
|---|---|---|---|
| ds DNA AT- | GCT TTA AAA AGT AAG TT | 10 | Double |
| rich | | | strand |
| ds22 | TTC GCG CGC GTT TTC GCG CGC G | 11 | Double |
| | | | strand |
| d(GC)8 | GCG CGC GCG CGC GCG C | 12 | Double |
| | | | strand |
| ds11 | CTA GGG CCT AG | 13 | Double |
| | | | strand |

ss DNA

| | | | |
|---|---|---|---|
| ss DNA T- | GGC CCT TTT TTT TCT AG | 14 | Single |
| rich | | | strand |
| s-myc | CCT TCC CCA CCC TCC CCA CCC TCC CCA | 15 | Single |
| | | | strand |
| s-Tel | CCC TAA CCC TAA CCC TAA CCC T | 16 | Single |
| | | | strand |
| Z-DNA | GCG CGC GCG CGC GCG C | 17 | Single |
| | | | strand |
| c-kit2_T15 | CGG GCG GGC GCG AGT GAG GGG | 18 | Single |
| | | | strand |
| DNA hairpin | CGC GGT GTC CGC G | 19 | Hairpin |

ss RNA

| | | | |
|---|---|---|---|
| RNA hairpin | GGA GAU CGC ACU CCA | 20 | Hairpin |

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### SELECTED EMBODIMENTS

Embodiment 1. A method for identifying G-quadruplexes (G4s) in a cell, the method comprising:
   (a) obtaining a fluorescent probe that selectively binds to G4s structures;
   (b) obtaining a cell comprising nucleic acids;
   (c) contacting the fluorescent probe with the nucleic acids in the cell;
   (d) detecting the fluorescence signal emitted when the fluorescent probe selectively binds to a G-quadruplex structure in the cell;
   (e) analyzing the fluorescence signal; and
   (f) identifying the G4 structures in the cell,
wherein the fluorescent probe is 2-(2-hydroxy-6-methoxy-3-propionylphenyl)quinazolin-4(3H)-one (HMPQ).
Embodiment 2. The method of embodiment 1, wherein the HMPQ utilized is selected from the group consisting of DNA grade, proteomics grade, molecular biology grade, and ultra-pure grade HMPQ.
Embodiment 3. The method of embodiment 1, wherein the polymorphic form of HMPQ utilized is conformer type 2.
Embodiment 4. The method of any of the preceding embodiments, wherein HMPQ binds to G4 structures in the cell with a peak emission wavelength from about 497 to about 500 nm.
Embodiment 5. The method of any of the preceding embodiments, wherein HMPQ binds to G4s in the cell with a peak emission wavelength at 500 nm.
Embodiment 6. The method of any of the preceding embodiments, wherein the nucleic acids are selected from the group consisting of DNA and RNA.
Embodiment 7. The method of any of the preceding embodiments, wherein HMPQ does not bind to nucleic acid structures that do not form G4s.
Embodiment 8. The method of any of the preceding embodiments, wherein HMPQ is substantially not cytotoxic towards mammalian cells and cell lines.
Embodiment 9. The method of any of the preceding embodiments, wherein HMPQ selectively binds to G4s without altering their conformation.
Embodiment 10. The method of any of the preceding embodiments, wherein HMPQ is compatible with use in aqueous media.
Embodiment 11. The method of any of the preceding embodiments, wherein the HMPQ limit of detection (LOD) is about 25 nM.
Embodiment 12. The method of any of the preceding embodiments, wherein the HMPQ binding to a G4 structure results in a fluorescence enhancement of up to about 300-fold at from about 497 to 500 nm.
Embodiment 13. The method of any of the preceding embodiments, wherein the HMPQ binding to G4 is detectable in subcellular cell compartments.
Embodiment 14. The method of embodiment 12, wherein the HMPQ binding to G4 is detectable in nucleus.
Embodiment 15. The method of any of the preceding embodiments, wherein the HMPQ is applied to a cell at a concentration of about 0.1 μM to about 1,000 μM.
Embodiment 16. The method of any of the preceding embodiments, wherein the method is label free.
Embodiment 17. A label-free kit for selectively detecting G-quadruplexes (G4s) in a cell, the kit comprising HMPQ of at least molecular biology grade and at least one of the following additional components: a cell fixation agent, a cell permeabilization agent, a cell fixation buffer, a cell permeabilization buffer, a washing buffer, or any combination thereof.
Embodiment 18. The kit of embodiment 17, wherein the cell fixation agent is 4% paraformaldehyde in PBS buffer, wherein the cell permeabilization is 0.2% Triton-X100 in PBS buffer, and wherein the washing agent is PBS buffer.

### REFERENCES

1. Ruggiero, E., & Richter, S. N. (2018). G-quadruplexes and G-quadruplex ligands: targets and tools in antiviral therapy. Nucleic acids research, 46(7), 3270-3283.
2. Chambers, V. S., Marsico, G., Boutell, J. M., Di Antonio, M., Smith, G. P., & Balasubramanian, S. (2015). High-throughput sequencing of DNA G-quadruplex structures in the human genome. Nature biotechnology, 33(8), 877-881.
3. Hänsel-Hertsch, R., Di Antonio, M., & Balasubramanian, S. (2017). DNA G-quadruplexes in the human genome: detection, functions and therapeutic potential. Nature reviews Molecular cell biology, 18(5), 279-284.
4. Li, Q., Xiang, J. F., Yang, Q. F., Sun, H. X., Guan, A. J., & Tang, Y. L. (2013). G4LDB: a database for discovering and studying G-quadruplex ligands. Nucleic acids research, 41(D1), D1115-D1123.
5. Mei, J., Leung, N. L., Kwok, R. T., Lam, J. W., & Tang, B. Z. (2015). Aggregation-induced emission: together we shine, united we soar!. Chemical reviews, 115(21), 11718-11940.
6. Hong, Y., Lam, J. W., & Tang, B. Z. (2011). Aggregation-induced emission. Chemical Society Reviews, 40(11), 5361-5388.
7. Mei, J., Hong, Y., Lam, J. W., Qin, A., Tang, Y., & Tang, B. Z. (2014). Aggregation-induced emission: the whole is more brilliant than the parts. Advanced materials, 26(31), 5429-5479.
8. Ding, D., Li, K., Liu, B., & Tang, B. Z. (2013). Bioprobes based on AIE fluorogens. Accounts of chemical research, 46(11), 2441-2453.
9. Wang, H., Li, Q., Alam, P., Bai, H., Bhalla, V., Bryce, M. R., . . . & Tang, B. Z. (2023). Aggregation-induced emission (AIE), life and health. ACS nano, 17(15), 14347-14405.
10. Hu, F, & Liu, B. (2016). Organelle-specific bioprobes based on fluorogens with aggregation-induced emission (AIE) characteristics. Organic & biomolecular chemistry, 14(42), 9931-9944.
11. Long, R., Tang, C., Xu, J., Li, T., Tong, C., Guo, Y., . . . & Wang, D. (2019). Novel natural myricetin with AIE and ESIPT characteristics for selective detection and imaging of superoxide anions in vitro and in vivo. Chemical Communications, 55(73), 10912-10915.
12. Cai, X. M., Lin, Y., Li, Y., Chen, X., Wang, Z., Zhao, X., . . . & Tang, B. Z. (2021). BioAIEgens derived from rosin: how does molecular motion affect their photophysical processes in solid state?. Nature communications, 12(1), 1773.
13. Nicolaou, K. C., Hale, C. R., Nilewski, C., & Ioannidou, H. A. (2012). Constructing molecular complexity and diversity: total synthesis of natural products of biological and medicinal importance. Chemical Society Reviews, 41(15), 5185-5238.
14. Shen, Y., Nie, C., Zhu, C., Zheng, Z., & Wu, Y. (2022). Aggregation-induced emission fluorophore-incorporated curcumin-based ratiometric nanoprobe for hypochlorite detection in food matrices. Journal of Agricultural and Food Chemistry, 70(30), 9577-9583.
15. He, T., Niu, N., Chen, Z., Li, S., Liu, S., & Li, J. (2018). Novel quercetin aggregation-induced emission luminogen (AIEgen) with excited-state intramolecular proton transfer for in vivo bioimaging. Advanced Functional Materials, 28(11), 1706196.
16. Gu, Y., Zhao, Z., Su, H., Zhang, P., Liu, J., Niu, G., . . . & Tang, B. Z. (2018). Exploration of biocompatible AIEgens from natural resources. Chemical science, 9(31), 6497-6502.
17. Chen, S. S., Wang, H., Wu, B., Li, Q., Gong, J., Zhao, Y. L., . . . & Tang, B. Z. (2023). Natural Coumarin Isomers with Dramatically Different AIE Properties: Mechanism and Application. ACS Central Science, 9(5), 883-891.
18. Nguyen, P. Q., Courchesne, N. M. D., Duraj-Thatte, A., Praveschotinunt, P., & Joshi, N. S. (2018). Engineered living materials: prospects and challenges for using biological systems to direct the assembly of smart materials. Advanced Materials, 30(19), 1704847.
19. Lane, A. L., & Moore, B. S. (2011). A sea of biosynthesis: marine natural products meet the molecular age. Natural product reports, 28(2), 411-428.
20. Berdy, J. (2005). Bioactive microbial metabolites. The Journal of antibiotics, 58(1), 1-26.
21. Montaser, R., & Luesch, H. (2011). Marine natural products: a new wave of drugs?. Future medicinal chemistry, 3(12), 1475-1489.
22. Hong, Y., Häußler, M., Lam, J. W., Li, Z., Sin, K. K., Dong, Y., . . . & Tang, B. Z. (2008).). Label-free fluorescent probing of G-quadruplex formation and real-time monitoring of DNA folding by a quaternized tetraphenylethene salt with aggregation-induced emission characteristics. Chemistry-A European Journal, 14(21), 6428-6437.
23. Wang, K. N., Liu, L. Y., Mao, D., Hou, M. X., Tan, C. P., Mao, Z. W., & Liu, B. (2022). A Nuclear-Targeted AIE Photosensitizer for Enzyme Inhibition and Photosensitization in Cancer Cell Ablation. Angewandte Chemie International Edition, 61(15), e202114600.
24. Qin, W., Alifu, N., Lam, J. W. Y., Cui, Y., SU. H., L. G., Qian, J., & Tang B. Z. (2020) Facile Synthesis of Efficient Luminogens with AIE Features for Three-Photon Fluorescence Imaging of the Brain through the Intact Skull. Advanced Materials, 32(23), e202000364.
25. Sedgwick, A. C., Wu, L., Han, H. H., Bull, S. D., He, X. P., James, T. D., . . . & Yoon, J. (2018). Excited-state intramolecular proton-transfer (ESIPT) based fluorescence sensors and imaging agents. Chemical Society Reviews, 47(23), 8842-8880.
26. Lipps, H. J., & Rhodes, D. (2009). G-quadruplex structures: in vivo evidence and function. Trends in cell biology, 19(8), 414-422.
27. Balasubramanian, S., & Neidle, S. (2009). G-quadruplex nucleic acids as therapeutic targets. Current opinion in chemical biology, 13(3), 345-353.
28. Cheng, A., Liu, C., Ye, W., Huang, D., She, W., Liu, X., & Qian, P. Y. (2022). Selective c9orf72 g-quadruplex-binding small molecules ameliorate pathological signatures of als/ftd models. Journal of Medicinal Chemistry, 65(19), 12825-12837.
29. Ye, W., Lui, S. T., Zhao, Q., Wong, Y. M., Cheng, A., Sung, H. H. Y., & Huang, P. (2023). Novel marine natural products as effective TRPVI channel blockers. International Journal of Biological Macromolecules, 253, 127136.
30. Dolomanov, O. V., Bourhis, L. J., Gildea, R. J., Howard, J. A., & Puschmann, H. (2009). OLEX2: complete structure solution, refinement and analysis program. Journal of applied crystallography, 42(2), 339-341.
31. Sheldrick, G. M. (2015). SHELXT-Integrated space-group and crystal-structure determination. Acta Crystallographica Section A: Foundations and Advances, 71(1), 3-8.
32. Bourhis, L. J., Dolomanov, O. V., Gildea, R. J., Howard, J. A., & Puschmann, H. (2015). The anatomy of a comprehensive constrained, restrained refinement program for the modern computing environment—Olex2 dissected. Acta Crystallographica Section A: Foundations and Advances, 71(1), 59-75.
33. Ye, W., She, W., Sung, H. Y., Qian, P., & Williams, I. D. (2020). Albofungin and chloroalbofungin: antibiotic crystals with 2D but not 3D isostructurality. Acta Crystallographica Section C: Structural Chemistry, 76(12), 1100-1107.
34. She, W., Ye, W., Cheng, A., Liu, X., Tang, J., Lan, Y., . . . & Qian, P. Y. (2021). Discovery, bioactivity evaluation, biosynthetic gene cluster identification, and heterologous expression of novel albofungin derivatives. Frontiers in Microbiology, 12, 635268.
35. KSHIRSAGAR, U. A. Recent developments in the chemistry of quinazolinone alkaloids. Organic & biomolecular chemistry, 2015, 13.36: 9336-9352.
36. Chen, X. W., Rao, L., Chen, J. L., & Zou, Y. (2022). Unexpected assembly machinery for 4 (3 H)-quinazolinone scaffold synthesis. Nature Communications, 13(1), 6522.
37. Wang, Z., Zhou, F., Wang, J., Zhao, Z., Qin, A., Yu, Z., & Tang, B. Z. (2018). Electronic effect on the optical properties and sensing ability of AIEgens with ESIPT process based on salicylaldehyde azine. Science China Chemistry, 61, 76-87.
38. Song, Z., Kwok, R. T., Zhao, E., He, Z., Hong, Y., Lam, J. W., . . . & Tang, B. Z. (2014). A ratiometric fluorescent probe based on ESIPT and ALE processes for alkaline phosphatase activity assay and visualization in living cells. ACS Applied Materials & Interfaces, 6(19), 17245-17254.
39. Zeng, Q., Li, Z., Dong, Y., Qin, A., Hong, Y., Ji, L., . . . & Tang, B. Z. (2007). Fluorescence enhancements of benzene-cored luminophors by restricted intramolecular rotations: AIE and AIEE effects. Chemical communications, (1), 70-72.
40. Yang, J., Ren, Z., Chen, B., Fang, M., Zhao, Z., Tang, B. Z., . . . & Li, Z. (2017). Three polymorphs of one luminogen: how the molecular packing affects the RTP and AIE properties?. Journal of Materials Chemistry C, 5(36), 9242-9246.
41. Liu, H., Lu, Z., Tang, B., Zhang, Z., Wang, Y., & Zhang, H. (2018). AIE-active organic polymorphs displaying molecular conformation-dependent amplified spontaneous emissions (ASE). Dyes and Pigments, 149, 284-289.
42. Huang, B., Chen, W. C., Li, Z., Zhang, J., Zhao, W., Feng, Y., . . . & Lee, C. S. (2018). Manipulation of molecular aggregation states to realize polymorphism, AIE, MCL, and TADF in a single molecule. Angewandte Chemie, 130(38), 12653-12657.
43. Parr, R. G., & Yang, W. (1995). Density-functional theory of the electronic structure of molecules. Annual review of physical chemistry, 46(1), 701-728.
44. Frisch, M. J., Trucks, G. W., Schlegel, H. B., Scuseria, G. E., Robb, M. A., Cheeseman, J. R., . . . & Fox, D. J. (2009). Gaussian 09, Revision D. 01, Gaussian, Inc., Wallingford CT. See also: URL: http://www.gaussian.com.
45. Duarte, A. R., Cadoni, E., Ressurreição, A. S., Moreira, R., & Paulo, A. (2018). Design of Modular G-quadruplex Ligands. ChemMedChem, 13(9), 869-893.
46. Murat, P., Singh, Y., & Defrancq, E. (2011). Methods for investigating G-quadruplex DNA/ligand interactions. Chemical Society Reviews, 40(11), 5293-5307.
47. Peterková, K., Durník, I., Marek, R., Plavec, J., & Podbevšek, P. (2021). c-kit2 G-quadruplex stabilized via a covalent probe: exploring G-quartet asymmetry. Nucleic Acids Research, 49(15), 8947-8960.
48. Gregory-Bryson, E., Bartlett, E., Kiupel, M., Hayes, S., & Yuzbasiyan-Gurkan, V. (2010). Canine and human gastrointestinal stromal tumors display similar mutations in c-KIT exon 11. BMC cancer, 10(1), 1-9.
49. Liu, Y., Tang, Y., Barashkov, N. N., Irgibaeva, I. S., Lam, J. W., Hu, R., . . . & Tang, B. Z. (2010). Fluorescent chemosensor for detection and quantitation of carbon dioxide gas. Journal of the American Chemical Society, 132(40), 13951-13953.
50. Wang, X., Han, T., Gong, J., Alam, P., Zhang, H., Lam, J. W., & Tang, B. Z. (2022). Diversity-Oriented Synthesis of Functional Polymers with Multisubstituted Small Heterocycles by Facile Stereoselective Multicomponent Polymerizations. Macromolecules, 55(11), 4389-4401.
51. Liu, C., Zhou, B., Geng, Y., Tam, D. Y., Feng, R., Miao, H., . . . & Zhu, G. (2019). A chair-type G-quadruplex structure formed by a human telomeric variant DNA in K+ solution. Chemical science, 10(1), 218-226.

We claim:

1. A method for identifying G-quadruplexes (G4s) in a cell, the method comprising:
(a) obtaining a fluorescent probe that selectively binds to G4s;
(b) obtaining a cell comprising nucleic acids;
(c) contacting the fluorescent probe with the nucleic acids in the cell;
(d) detecting a fluorescence signal emitted when the fluorescent probe selectively binds to a G4 structure in the cell;
(e) analyzing the fluorescence signal; and
(f) identifying the G4 structure in the cell,
wherein the fluorescent probe is 2-(2-hydroxy-6-methoxy-3-propionylphenyl)quinazolin-4(3H)-one (HMPQ).

2. The method of claim 1, wherein the HMPQ utilized is selected from the group consisting of DNA grade, proteomics grade, molecular biology grade, and ultra-pure grade HMPQ.

3. The method of claim 1, wherein a polymorphic form of the HMPQ utilized is conformer type 2.

4. The method of claim 1, wherein the HMPQ binds to a G4 structure in the cell with a peak emission wavelength from about 497 nm to about 500 nm.

5. The method of claim 1, wherein the HMPQ binds to a G4 structure in the cell with a peak emission wavelength at 500 nm.

6. The method of claim 1, wherein the nucleic acids are selected from the group consisting of DNA and RNA.

7. The method of claim 1, wherein the HMPQ does not bind to nucleic acid structures that do not form G4s.

8. The method of claim 1, wherein the HMPQ is substantially not cytotoxic towards mammalian cells and cell lines.

9. The method of claim 1, wherein the HMPQ binds to G4s without altering their conformation.

10. The method of claim 1, wherein the HMPQ is compatible with use in aqueous media.

11. The method of claim 1, wherein the HMPQ limit of detection (LOD) is about 25 nM.

12. The method of claim 1, wherein the HMPQ binding to a G4 structure results in a fluorescence enhancement of up to about 300-fold at from about 497 nm to 500 nm.

13. The method of claim 1, wherein the HMPQ binding to G4 is detectable in subcellular cell compartments.

14. The method of claim 13, wherein the HMPQ binding to G4 is detectable in nucleus.

15. The method of claim 1, wherein the HMPQ is applied to a cell at a concentration of about 0.1 μM to about 1,000 μM.

16. The method of claim 1, wherein the method is label free.

17. A label-free kit for selectively detecting G-quadruplexes (G4s) in a cell, the kit comprising HMPQ of at least molecular biology grade and at least one of the following additional components: a cell fixation agent, a cell permeabilization agent, a cell fixation buffer, a cell permeabilization buffer, a washing buffer, or any combination thereof.

18. The kit of claim 17, wherein the cell fixation agent is 4% paraformaldehyde in PBS buffer, wherein the cell permeabilization is 0.2% Triton-X100 in PBS buffer, and wherein the washing agent is PBS buffer.


SEQUENCE LISTING (20 sequences)

SEQ ID NO: 1 - DNA, 22 bp, synthetic construct
  source 1..22: mol_type = other DNA
tgagggtgggtagggtgggtaa

SEQ ID NO: 2 - DNA, 22 bp, synthetic construct
  source 1..22: mol_type = other DNA
agggagggcgctgggaggaggg

SEQ ID NO: 3 - DNA, 21 bp, synthetic construct
  source 1..21: mol_type = other DNA
cgggcgggcgcgagggagggg

SEQ ID NO: 4 - DNA, 23 bp, synthetic construct
  source 1..23: mol_type = other DNA
tagggttagggttagggttgggg

SEQ ID NO: 5 - DNA, 28 bp, synthetic construct
  source 1..28: mol_type = other DNA
gggaggcgtggcctgggcgggactgggg

SEQ ID NO: 6 - DNA, 21 bp, synthetic construct
  source 1..21: mol_type = other DNA
gggttagggttagggtttggg

SEQ ID NO: 7 - DNA, 24 bp, synthetic construct
  source 1..24: mol_type = other DNA
ggggccggggccggggccggggcc

SEQ ID NO: 8 - RNA, 12 nt, synthetic construct
  source 1..12: mol_type = other RNA
ggggccggggcc

SEQ ID NO: 9 - RNA, 12 nt, synthetic construct
  source 1..12: mol_type = other RNA
tagggttagggt

SEQ ID NO: 10 - DNA, 17 bp, synthetic construct
  source 1..17: mol_type = other DNA
gctttaaaaagtaagtt

SEQ ID NO: 11 - DNA, 22 bp, synthetic construct
  source 1..22: mol_type = other DNA
ttcgcgcgcgttttcgcgcgcg

SEQ ID NO: 12 - DNA, 16 bp, synthetic construct
  source 1..16: mol_type = other DNA
  misc_feature 1..16: note = double strand
gcgcgcgcgcgcgcgc

SEQ ID NO: 13 - DNA, 11 bp, synthetic construct
  source 1..11: mol_type = other DNA
ctagggcctag

SEQ ID NO: 14 - DNA, 17 bp, synthetic construct
  source 1..17: mol_type = other DNA
ggcccttttttttctag

SEQ ID NO: 15 - DNA, 27 bp, synthetic construct
  source 1..27: mol_type = other DNA
ccttccccaccctccccaccctcccca

SEQ ID NO: 16 - DNA, 22 bp, synthetic construct
  source 1..22: mol_type = other DNA
ccctaaccctaaccctaaccct

SEQ ID NO: 17 - DNA, 16 bp, synthetic construct
  source 1..16: mol_type = other DNA
  misc_feature 1..16: note = single strand
gcgcgcgcgcgcgcgc

SEQ ID NO: 18 - DNA, 21 bp, synthetic construct
  source 1..21: mol_type = other DNA
cgggcgggcgcgagtgagggg

SEQ ID NO: 19 - DNA, 13 bp, synthetic construct
  source 1..13: mol_type = other DNA
cgcggtgtccgcg

SEQ ID NO: 20 - RNA, 15 nt, synthetic construct
  source 1..15: mol_type = other RNA
ggagatcgcactcca